(19) United States
(12) Patent Application Publication
(10) Pub. No.: US 2011/0046540 A1
(43) Pub. Date: Feb. 24, 2011

(54) Apparatus for Trans-Cerebral Electrophoresis and Methods of Use Thereof

(76) Inventors: Ron L. Alterman, New York, NY (US); Jay L. Shils, Reading, MA (US)

Correspondence Address: KING & SPALDING, 1185 AVENUE OF THE AMERICAS, NEW YORK, NY, 10036-4003, US

(21) Appl. No.: 12/862,236

(22) Filed: Aug. 24, 2010

Related U.S. Application Data
(60) Provisional application No. 61/236,303, filed on Aug. 24, 2009.

(51) Int. Cl. A61N 1/30

(52) U.S. Cl. 604/21; 604/501

(57) ABSTRACT

The present invention provides apparatus and methods for the delivery of therapeutic agents to target tissues by electromigration. The utilization of electric fields according to the methods of the invention aids in the distribution and targeting of therapeutic agents, in particular, where standard means of agent application in the target tissue is insufficient to achieve prophylactic or therapeutic results. In particular embodiments, the present invention utilizes a convective force in combination with the developed electric fields to further increase the flux of the therapeutic agent or to further improve distribution of the therapeutic agent within the target tissues.

This application claims priority benefit, under 35 U.S.C. §119(e), of U.S. Provisional Patent Application 61/236,303, filed Aug. 24, 2009, the contents of which application are hereby incorporated by reference in their entirety.

### 1. INTRODUCTION

The present invention provides apparatus and methods for the delivery of therapeutic agents to target tissues through the use of electric fields. The utilization of electric fields according to the methods of the invention aids in the distribution and targeting of therapeutic agents, in particular, where standard means of agent application is insufficient or impractical to reach target tissues. In particular embodiments, the present invention utilizes a convective force in combination with the developed electric fields to further increase the flux or distribution of the therapeutic agent within the target tissues.

### 2. BACKGROUND OF THE INVENTION

The ability to treat neurological and mental disorders is limited, in part, by the ability to deliver therapeutic compounds efficiently to the brain parenchyma. Pharmaceuticals are delivered to the majority of bodily tissues via the oral (PO), intramuscular (IM) or intravenous (IV) routes. When these mechanisms prove insufficient, intra-arterial delivery via selective catheterization can be used to achieve high local concentrations of the desired agent while limiting the risks of systemic toxicity. For bone disorders, intramedullary delivery can achieve the same results. These delivery routes are relatively ineffective for disorders of the central nervous system (CNS) because of the blood-brain barrier (BBB). The BBB, which is specific to the brain and spinal cord and is formed by tight junctions that bind adjoining cerebral endothelial cells, protects the CNS from toxic substances that may enter the bloodstream from time to time. Unfortunately, the BBB also impedes the delivery of therapeutic agents via the blood, creating unique difficulties for treating most neurological disorders. Although numerous strategies have been devised to disrupt or bypass the BBB, the lack of clinically relevant methods indicates that improved methods are necessary.

As one method of bypassing the BBB, neuroscientists have turned to direct intracerebral infusion, which requires the surgical implantation of microcatheters that effect infusion of therapeutic agents into specific brain regions. The catheters are connected to pumps, which deliver the desired agent. The most promising form of parenchymal infusion is termed “convection enhanced drug delivery” or CEDD. Developed more than a decade ago at the NIH, CEDD employs steady positive pressure to push macromolecules through the neuropil slowly and atraumatically (see, e.g., Bobo, et al., 1994, PNAS USA 91:2076-80). This technique has been demonstrated to be effective for infusions into relatively small target areas and these types of pumps are currently in use in a number of prospective clinical trials. However, even if CEDD proves effective in delivering therapeutic agents to small tissue volumes, the technique may not be scalable, leaving numerous brain disorders that may require treatment of much greater tissue volumes unaffected. For example, reported failures of CEDD infusions of glial derived neurotropic factor (GDNF) for the treatment of Parkinson's disease may have been caused by inadequate distribution of the GDNF infusate within the putamen rather than a failure of the GDNF to generate the desired biological effect. Thus, while CEDD represents significant progress toward a viable intracerebral drug delivery system, there is a great need for an improved drug delivery system so that a greater variety of neurological and psychiatric disorders can be treated.

### 3. SUMMARY OF THE INVENTION

The invention is directed to apparatus, and methods of use thereof, for the delivery of therapeutic, diagnostic or investigational agents to target tissues of a subject in need thereof. In specific embodiments, the invention is directed to apparatus for the delivery of agents to target tissue which apparatus may be used in combination with or is itself part of a second apparatus or system for providing an electric field within a target tissue to effect the distribution and/or targeting of agents within the tissue. The application of an electric field to target tissues as described herein is termed trans-cerebral electrophoresis (“TCE”). In specific embodiments, the target tissue is tissue of the central nervous system (“CNS”) and, in particular, tissue of the brain or spinal cord. Without being bound by a particular mechanism of action, it is believed that the application of an electric field within the target tissue results in an electromotive force (EMF) to the one or more agents (e.g., one or more therapeutic, investigational, and/or diagnostic agents) that improves or modifies dispersive forces normally present in the tissue, e.g., diffusive distribution. This invention is also directed to the methods for use of the apparatus described herein for providing one or more agents to target tissues. In specific embodiments, the one or more agent is an agent for the treatment or diagnosis of a disease or disorder of the CNS, and the target tissue is the situs of the disease or disorder. In other embodiments, the methods of the invention are used in connection with investigations of central nervous system function.

The invention provides for an integrated TCE cannula for delivery of a fluid to a tissue delivery site of a subject, which integrated TCE cannula comprises an implantable cannula having proximal and distal ends, a fluid delivery pathway through the cannula and, at its distal end, 1) one or more outlet ports for the fluid pathway through which the agent is administered to the delivery site and 2) one or more monopolar electrodes having a region for electrical connection to a power source. When the fluid delivery pathway of the cannula is in fluid connection or communication with a fluid delivery system, the fluid flows through the delivery pathway of the cannula and exits via the outlet ports into the tissue delivery site. In certain embodiments, the integrated TCE cannula comprises, at its proximal end, a connector for connecting the fluid delivery pathway with a fluid delivery system and/or a connector for connecting the monopolar electrode to a power source. The connectors may be suitable to allow temporary communication between the integrated TCE cannula and the fluid delivery system and/or power source (i.e., allowing the cannula to be readily disconnected from the fluid delivery system and/or power source, or allowing multiple integrated TCE cannulas to be used sequentially with a single fluid delivery system and/or power source) or may be such that the connection between the integrated TCE cannula and the fluid delivery system and/or power source is permanent. In preferred embodiments, the fluid comprises one or more of a therapeutic, diagnostic or investigational agent in a pharmaceutically acceptable carrier. In certain embodiments, the integrated TCE cannula further comprises thermocouple in contact with the electrode, cannula and/or surrounding tissue. In certain embodiments, thermocouple is in communication with a display device for display of the temperature of the cannula, electrode and/or surrounding tissue and/or may further be connected to a processor for automatic regulation of the parameters of the TCE or agent delivery as described herein. The integrated TCE cannula may be disposable in that it is designed for a single use or may be designed for repeated use. In embodiments where the integrated TCE cannula is to be reused, the materials of the cannula are suitable for sterilization by any method known in the art.

The integrated TCE cannula of the invention is suitable for the direct infusion of fluids into the body tissues of a subject in need thereof, and, in specific embodiments, is suitable for convective enhanced drug delivery into the tissue of the central nervous system. In certain embodiments, the integrated TCE cannula is a reflux-free cannula. To this end, in certain embodiments, the TCE cannula is in communication with an agent delivery system suitable for delivery of one or more agents to the tissue of the patient via the cannula. In certain embodiments, the invention encompasses apparatus comprising, in addition to the integrated TCE cannula, an agent delivery system comprising one or more pumps that provide one or more agents, e.g., one or more therapeutic, investigational, or diagnostic agents, to the integrated TCE cannula and, thus, to the delivery area within the subject's tissues. In certain embodiments, the invention encompasses the use of one or more integrated TCE cannulas for introduction of one or more agents to the delivery area within the tissues of the subject, e.g., tissues of the CNS. The invention may further comprise an agent delivery system comprising one or more regulators that control the agent delivery via the one or more integrated TCE cannulas so as to supply a specified total dose and/or to supply a specified agent delivery rate. The one or more integrated TCE cannulas and/or agent delivery systems may be designed for temporary use or permanent implantation as is known in the art. For example, the integrated TCE cannulas may be designed to allow insertion into the tissues of the subject without external housings, e.g., in the manner of an syringe needle, or may be designed to comprise external housings that aid insertion, which housings are withdrawn leaving the cannula implanted within the delivery area. In certain embodiments, the cannulas may be pre-filled with a therapeutic, diagnostic, or investigational agent and/or with a pharmaceutical carrier prior to implantation/insertion. In certain embodiments, the one or more integrated TCE cannulas and agent delivery system (including, but not limited to, any pumps, agent reservoirs and regulators) may be fully implantable as is known in the art. As used herein, the term “cannula” and “integrated TCE cannula” encompasses the device through which an agent is provided to the delivery area within the tissue or tissues of the patient, and thus may encompass a variety of materials, designs and sizes depending on the delivery area, including, but not limited to, inflexible needles/tubing and flexible tubing devices, as is well known and routinely implemented in the art.

The monopolar electrode of the integrated TCE cannula may be used in conjunction with one or more, e.g., an array or a plurality, of independently polarizable monopolar electrodes to generate an electric field that at least partially encompasses the target tissue. Accordingly, in certain embodiments, in addition to the integrated TCE cannula, the apparatus of the invention comprises one or more monopolar electrodes, separate from that of the integrated TCE cannula, each of which monopolar electrode is independently polarizable. The array of monopolar, polarizable electrodes, each in connection with a power source (e.g., a current and/or voltage source), when powered, effects the generation of the electric field at least partially encompassing the target tissue. In certain embodiments the electrodes comprises a connector suitable for connection to a power source, which connection may be temporary or permanent. The array includes at least two electrodes such that when connected to the power source and polarized, an electric field is generated between the two or more electrodes. In specific embodiments, the array of electrodes comprises two or more or a plurality of electrodes, one of which is the electrode of the integrated TCE cannula. The remaining electrodes that form the array may be surface or implantable electrodes. In specific embodiments, the invention provides a spatial arrangement of electrodes, such that, when the array is connected to the power source, an electric field is generated that at least partially encompasses the target tissue. Application or administration of one or more agents (e.g., one or more therapeutic or diagnostic agents) within a suitably oriented electric field will cause the agent(s) that respond to electric fields, i.e., charged or ionized agents, to move down the electric gradient, preferably, to or within the target tissue.

The array of electrodes comprises at least two electrodes, one of which may be the electrode of the integrated TCE cannula, and may comprise any number sufficient for development of the desired electric field within the target tissue. In certain embodiments, the array of electrodes comprises at least 2, at least 3, at least 4, at least 5, at least 6, at least 7, at least 8, at least 9, at least 10, at least 11 or at least 12 electrodes. In other embodiments, the array of electrodes comprises no more than 2, no more than 3, no more than 4, no more than 5, no more than 6, no more than 7, no more than 8, no more than 9, no more than 10, no more than 11 or no more than 12 electrodes. In preferred embodiments, the array of electrodes comprises from 2 to 5 electrodes. In yet more preferred embodiments, the array of electrodes comprises 3 to 4 electrodes. In the preferred embodiments, at least one of the electrodes in the array is the electrode of the integrated TCE cannula.

The electrode array of the invention comprises a sufficient number of electrodes placed in a suitable three-dimensional (“3D”) orientation such that, when connected to the power source, an electric field is developed in the array, which electric field at least partially encompasses the target tissue. The electrodes may or may not be in contact with the subject, e.g., in certain embodiments, the apparatus of the invention comprises external electrodes which electrodes are not in direct contact with the subject and/or target tissue. In alternate embodiments, the apparatus of the invention comprises an electrode array in direct contact with the subject and/or target tissue (e.g., placed on or placed/implanted within the subject or target tissue). In a specific example in accordance with this embodiment, the apparatus of the invention comprises an array of implantable and/or surface electrodes. In certain embodiments, the apparatus of the invention comprises an array of surface electrodes. In other embodiments, the apparatus of the invention comprises an array of implantable electrodes. In yet other embodiments the apparatus of the invention comprises an array of surface and implantable electrodes.

In specific embodiments of the invention, the electric field developed within the electrode array encompasses the site of administration (i.e., the delivery area) of the one or more therapeutic or investigational agents. In other embodiments, the electric field developed by the electrode array does not encompass the delivery area and the one or more agents enter the electric field by dispersive forces within the target tissue of the subject (e.g., via diffusion, active transport, bulk flow, blood flow, etc.).

In certain embodiments of the invention, the invention encompasses an apparatus comprising a power source and, in some embodiments, further comprising one or more regulators for regulating and/or controlling the power provided to the individual electrodes within the electrode array. The power source and/or power source and regulator may provide a current and/or voltage to the array such that the developed electric field maintains a constant strength and/or polarity throughout the entirety of a TCE session. In alternate embodiments, the power source and/or power source and regulator provide a current and or voltage to the array such that the developed electric field is variable in strength and/or polarity over a single TCE session. The power source and/or power source and regulator may provide a direct or alternating current. The power provided to the electrode array (e.g., the current) may be continuous or pulsed.

The power supplied to the electrode array is sufficient to effect the dispersion of the therapeutic, investigational and/or diagnostic agent to or within the target tissue. In preferred embodiments, the power supplied to the electrode array is below the threshold level to effect electroporation of the agent within the target tissue. In a specific example in accordance with this embodiment, the developed electric gradient within the array is less than 100 kV/cm. In other examples, the developed electric gradient is less than 10 kV/cm or less than 5 kV/cm. In still other examples the developed electric gradient is less than 95, 90, 85, 80, 75, 70, 65, 60, 55, 50, 45, 40, 35, 30, 25, 20, or 15 kV/cm.

In certain embodiments, invention comprises the use of one or more biosensors. The biosensors may be separate components of the apparatus of the invention or may be integrated into one or more other components of the apparatus in contact with the subject, e.g., incorporated into the one or more electrodes or the one or more integrated TCE cannulas. The biosensors of the apparatus can monitor one or more performance parameters of the apparatus (e.g., agent delivery rate, electric field strength) and/or one or more patient specific parameters (e.g., temperature of the tissue surrounding the two or more electrodes within the array, or local pressure or pressure gradients within the tissue surrounding the one or more agent delivery cannulas). The biosensors of the apparatus of the invention may comprise connectors for connecting to external display devices allowing manual regulation of apparatus function in response to the displayed output of the one or more biosensors. In other embodiments, the biosensors of the apparatus of the invention comprise connectors for communication with a processor that automatically regulates apparatus operation in response to signals from the one or more biosensors.

The methods of the invention can be used with any method of agent (e.g., drug) delivery known in the art that is suitable for administration of an agent to the delivery area within the tissues of the subject. In other embodiments, the methods of the invention encompass administration of an agent to the subject within the developed electric field. In alternate embodiments, the methods of the invention encompass administration of an agent external to the developed electric field, which agent then enters the electric field by dispersive forces acting at the site of administration other than the developed EMF. In certain embodiments, the invention encompasses the use of convection enhanced drug delivery (“CEDD”) for administration of an agent. Such convection enhanced methods are well known in the art and are routinely used to provide, for example, therapeutic or diagnostic agents to CNS tissues under pressure. It is believed that methods of the invention combining TCE and CEDD will not only improve agent delivery (i.e., therapeutic or diagnostic agent delivery) to target tissues (e.g., tissues of the CNS), but also allow targeting of an agent that is not possible using current methods known in the art. Because the methods of the invention use EMF to direct the one or more agents within the tissue, existing limitations of CEDD may be overcome. In certain embodiments, agent administration need not be direct or near target tissue, but can be in a more remote site. Such embodiments are advantageous, particularly, for example, wherein direct access via traditional administration methods (e.g., injection, cannulation, catheterization) would be impractical or impossible.

In certain embodiments, the integrated TCE cannulas of the invention are suitable for agent administration in accordance with the methods of CEDD as known in the art. The integrated TCE cannula of the invention combines the function of an infusion catheter for CEDD and one or more electrophoretic electrodes. Having at least one of the polarizable electrodes of the electrode array at the site of agent administration allows the developed electric field to be modulated to better focus, direct and/or regulate agent dispersal to or within the target site. As described herein, in addition to the integrated TCE cannula, the invention comprises one or more monopolar electrodes separate from that of the implantable, integrated TCE cannula having a connector for connecting to a power source. When the array of electrodes is connected to the power source and separately polarized, the electric field is generated by the array. The invention also encompasses methods of use of the apparatus described herein for delivery of an agent to a target tissue within the CNS of a subject, e.g., for the treatment, prevention or amelioration of one or more symptoms of a CNS disease or disorder.

The invention also comprises a method for delivering an agent to or within a target tissue of the CNS of a subject, said method comprising the steps of A) positioning an array of electrodes such that, when powered and separately polarized, the array is positioned so as to provide an electric field of sufficient amplitude and polarity to cause movement of the agent from the delivery area (or point of entry of the agent within the field) to or within the target tissue of the subject; B) polarizing the array of electrodes and thereby generating an electric field in the array; and C) applying the agent to the delivery area. In specific embodiments, the methods of the invention comprise applying the agent to the delivery area, which delivery area is within the electric field developed by the powered electrode array. In certain embodiments, the agent is applied to the delivery area prior to, concomitant with, or subsequent to the powering of the electrode array. The spatial arrangement of the electrodes in the array causes the target tissue to be at least partially encompassed by the electric field, and the electric field provides an EMF to drive the one or more agents to or within the target tissue. In preferred embodiments, the target tissue is tissue of the CNS and the electrode array is positioned such that, when powered, the developed EMF provides a dispersive force within the CNS tissue, along the surface of the CNS tissue, within the subcutaneous tissue surrounding the CNS tissue, or on the surface/within the skin of the subject. In specific embodiments, the method of the invention encompasses the treatment, prevention or amelioration of one or more symptoms of a disease or disorder of the CNS in a subject in need thereof.

Therapeutic agents for use in accordance with the methods of the invention include any agent that will migrate along an electric potential gradient (i.e., charged molecules, dipoles). Such therapeutics may naturally respond to an EMF or can be modified to respond provided that the modification does not alter their desired bioactivity

3.1 Terminology

As used herein, the term “about” or “approximately” when used in conjunction with a number refers to any number within 1, 5 or 10% of the referenced number or within the experimental error typical of standard methods used for the measurement and/or determination of said number.

As used herein, the term “central nervous system (‘CNS’) disorder” and analogous terms refer to a disorder associated with the death and/or dysfunction of a particular neuronal or non-neuronal cell population (e.g., glial cells) in the CNS and/or the aberrant growth of cells within the CNS. The aberrantly growing cells of the CNS may be native to the CNS or may be derived from other tissues, and may be malignant or non-malignant. The disorder may be acute or chronic. Non limiting examples of CNS disorders include, but are not limited to, cancer, neoplastic growth, infection, head trauma, spinal cord injury, multiple sclerosis, dementia with Lewy bodies, ALS, lysosomal storage disorders, amyloidogenic diseases (e.g., Alzheimer's disease), neurodegenerative diseases, autoimmune disorders, stroke, epilepsy, psychiatric disorders, and disorders of hormonal balance. Further contemplated are methods for reducing inflammation that is associated with a CNS disorder characterized by neuronal death and/or dysfunction.

As used herein, the term “in combination” in the context of the administration of (a) therapy(ies) to a subject, refers to the use of more than one therapy (e.g., more than one prophylactic and/or therapeutic agent or method). The use of the term “in combination” does not restrict the order in which therapies (e.g., prophylactic and/or therapeutic agents or methods) are administered to a subject, but instead refers to the use of more than one therapy as part of an overall treatment regimen. A first therapy (e.g., a first prophylactic and/or therapeutic agent or method) can be administered prior to (e.g., at least 5 minutes, at least 15 minutes, at least 30 minutes, at least 45 minutes, at least 1 hour, at least 2 hours, at least 4 hours, at least 6 hours, or at least 12 hours before), concomitantly with, or subsequent to (e.g., at least 5 minutes, at least 15 minutes, at least 30 minutes, at least 45 minutes, at least 1 hour, at least 2 hours, at least 4 hours, at least 6 hours, or at least 12 hours after) the administration of a second therapy (e.g., a second prophylactic and/or therapeutic agent or method) to a subject.

As used herein, the terms “manage,” “managing,” and “management” refer to the beneficial effects that a subject derives from a therapy (e.g., a prophylactic and/or therapeutic agent or method), which does not result in a cure of the disease or disorder, e.g., a CNS disease or disorder. In certain embodiments, a subject is administered one or more therapies (e.g., prophylactic and/or therapeutic agents or methods) to “manage” a condition or symptom associated with a disease or disorder (e.g., a CNS disease or disorder), so as to prevent the progression or worsening of the disease/disorder.

As used herein, the terms “prevent”, “preventing” and “prevention” refer to the prevention of onset of, the recurrence of, or a reduction in one or more symptoms of a disease/disorder (e.g., disorder of the CNS) in a subject as a result of the administration of a therapy (e.g., a prophylactic and/or therapeutic method of the invention).

As used herein, the terms “therapies” and “therapy” can refer to any protocol(s), method(s), and/or agent(s) that can be used in the diagnosis, prevention, treatment, management, or amelioration of a disease/disorder, and/or a symptom thereof (e.g., a CNS disease or disorder or a condition or symptom associated therewith). In certain embodiments, the terms “therapies” and “therapy” refer to diagnostic procedures, biological therapy, supportive therapy, and/or other therapies useful in diagnosis, treatment, management, prevention, or amelioration of a disease or condition, or of one or more symptoms associated therewith.

As used herein, the terms “treat,” “treatment,” and “treating” in the context of administration of a therapy to a subject for a disease or disorder refers to the cure of the disease or disorder, or may refer to the eradication, reduction or amelioration of one or more symptoms of said disease/disorder (e.g., CNS disease/disorder).

### 4. DESCRIPTION OF THE FIGURES

FIG. 1 Schematic of exemplary integrated TCE cannula

FIG. 1A Schematic of a cross section of the exemplary integrated TCE cannula of FIG. 1.

FIG. 1B Schematic of an electrode portion of the exemplary integrated TCE cannula of FIG. 1.

FIG. 2 Schematic of exemplary distal end of the TCE cannula

FIG. 3 Schematic of exemplary arrangement of electrode array

FIG. 4 Schematic of exemplary arrangement of fluid regulating components of the TCE apparatus.

FIG. 5 Schematic of exemplary control circuit for an individual electrode and/or electrode lead.

### 5. DETAILED DESCRIPTION OF THE INVENTION

The invention provides for the use of an electric field to effect the distribution and/or the targeting of charged agents within a target tissue, such as that of the CNS. The application of an electric field within the tissue results in an electromotive force (EMF) that disperses or moves the agent to or within the target tissue. The movement or dispersal provided by the EMF according to the methods of the invention may also be used to improve or modify the movement associated with other dispersive forces, e.g., those associated with diffusive distribution or convective enhanced drug delivery (“CEDD”).

In particular embodiments, the methods of the invention provide for the use of electrophoresis in combination with convective enhanced drug delivery (“CEDD”). The addition of an electromotive force (EMF) to agents represents a major improvement to CEDD. Charged molecules, including proteins and nucleic acids, can be directed along a potential gradient so long as the appropriate electrical field is created between or among the two or more electrodes. Clinical use of CEDD has demonstrated that the tissue of the CNS is, in fact, a porous matrix that permits the flow of macromolecules through the matrix without damage to cytoarchitecture or induction of neurological deficits. Application of a low level electric field across or including the target tissue will create a potential gradient down which the applied or introduced agent(s) (e.g., therapeutic, diagnostic, or investigative agents) will migrate. Employed over a period of days, weeks, months or years, the charge gradient will enhance the treatment volume of parenchymal infusions, dramatically increasing their potential clinical applications.

The central nervous system can function well despite the application of low-level, therapeutic, exogenous electrical current. For example, chronic spinal cord stimulation has become a mainstay of chronic pain management, allowing patients with otherwise disabling pain syndromes to lead fuller lives without any untoward effects from the stimulation on normal spinal cord functions. Vagal nerve stimulation has proven to be an effective treatment for generalized epilepsy when medications fail to provide adequate seizure control and surgical resection of the seizure focus is not feasible. Also, deep brain stimulation (“DBS”) has become the treatment of choice for movement disorders such as Parkinson's disease, Essential Tremor, and Idiopathic Torsion Dystonia when medications fail to provide adequate symptomatic relief. In all instances, the low level electric fields developed during these therapies are well-tolerated. However, unlike these highly localized therapies, the instant invention utilizes trans-cerebral electrophoresis (“TCE”): the creation of a relatively larger electric field not to stimulate or lesion a discrete region of tissue, e.g., excitable tissue, but to create an electric gradient of defined shape and volume down which therapeutic agents will migrate.

In certain embodiments, TCE according to the methods of the invention enhances the efficacy of parenchymal infusion, e.g., CEDD, by broadening the distribution of an infused agent such as a therapeutic, investigational or diagnostic agent. In other embodiments, TCE according to the methods of the invention enhances the efficacy of parenchymal infusion, e.g., CEDD, by allowing targeting to specific tissues or specific volumes of tissue. For example, the methods of the invention allow the parameters of the parenchymal infusion and TCE to vary to achieve specific tissue distribution goals. For example, the methods of the invention allow a single application of a therapeutic agent directly to target tissues in conjunction with TCE to distribute the agent over a larger volume of target tissue than a standard single application (e.g., via diffusion or CEDD) would allow. In other embodiments, the methods of the invention allow application of an agent at a site remote to the target tissue (e.g., where direct application is impossible or impractical), and the use of TCE to establish an electric gradient that directs the agent to the target tissue. In still other embodiments, the methods of the invention allow the distribution of the therapeutic agent to be controlled such that only a desired volume, shape or area of target tissue is contacted by the agent.

In certain embodiments, the invention provides for the treatment, management or prevention of a CNS disease or disorder or for the treatment, management, prevention or amelioration of one or more symptoms of a CNS disease or disorder. In certain examples in accordance with this embodiment, the disease or disorder is a neurodegenerative disease, neurodegeneration associated with stroke, neurodegeneration associated with cancer or a disease or disorder associated with neuronal death and/or dysfunction. Non-limiting examples of CNS disorders include, but are not limited to, cancer, neoplastic growth, infection, head trauma, spinal cord injury, multiple sclerosis, dementia with Lewy bodies, ALS, lysosomal storage disorders, amyloidogenic diseases (e.g., Alzheimer's disease), neurodegenerative diseases, autoimmune disorders, tauopathies, stroke, epilepsy, psychiatric disorders, and disorders of hormonal balance. Further contemplated are methods for reducing inflammation that is associated with a CNS disorder characterized by neuronal death, infection, and/or dysfunction.

In certain embodiments, the invention provides for the treatment, management or prevention of a CNS cancer or for the treatment, management, prevention or amelioration of one or more symptoms of a CNS cancer in a subject in need thereof. The CNS cancer may be a cancer originating from CNS cells or may include tumor(s) derived from cells of other tissues of the body, e.g., a metastasized tumor(s) to the CNS. The methods of the invention encompass the direct application of therapeutic agents to the tumor(s) (e.g., at or on the surface of, or within the tumor) or, alternatively, application at a site distal to the tumor wherein TCE is used to regulate, control, or direct the therapeutic agent to and/or within the tumor site.

In certain embodiments, the invention provides for the diagnosis or investigation of a CNS disease or disorder comprising the administration of a diagnostic or investigational agent. In certain embodiments, the diagnostic agent or investigational agent may comprise a targeting moiety that targets the agent to specific cell types or that causes the preferential uptake of the agent within a specific cell population. In other embodiments, the diagnostic or investigational agent is a contrast agent suitable for use with tissue visualization modalities such as, but not limited to, X-ray, Computerized Tomography (CT), magnetic resonance imaging (MRI), optical imaging, positron emission tomography (PET scanning) or Single Photon Emission Computerized Tomography (SPECT). In specific examples, the diagnostic agent or investigational agent comprises an antibody or antigen binding fragment thereof specific for a tumor, neoplastic and/or malignant cell marker, which antibody when used in accordance with the methods of the invention allows the detection and localization of cells expressing the tumor, neoplastic and/or malignant marker. The methods of the invention encompass the use of diagnostic or investigational agents, for example, to detect the presence or absence of a disease, disorder or infection (or to detect characteristic indicators thereof), or to monitor the development or progression of a disease, disorder or infection as part of a clinical testing procedure to, e.g., determine the efficacy of a given treatment regimen. Diagnostic or investigational agents for use in accordance with the methods of the invention may respond themselves to the developed EMF (i.e., the agent is itself a charged molecule or dipole), or may be conjugated to a molecule that exhibits such activity (i.e., acting as a carrier for the diagnostic or investigational molecule and/or that targets the diagnostic molecule to a tissue of interest). In specific embodiments, the diagnostic or investigational agent is coupled to a detectable substance to aid in detection of the agent. Non-limiting examples of detectable substances include, but are not limited to, various enzymes, including, but not limited to, horseradish peroxidase, alkaline phosphatase, beta-galactosidase, or acetylcholinesterase); prosthetic group complexes, such as, but not limited to, streptavidin/biotin and avidin/biotin; fluorescent materials, such as, but not limited to, umbelliferone, fluorescein, fluorescein isothiocyanate, rhodamine, dichlorotriazinylamine fluorescein, dansyl chloride or phycoerythrin; luminescent materials, such as, but not limited to, luminol; bioluminescent materials such as, but not limited to, luciferase, luciferin, and aequorin; radioactive material, such as, but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

Any type of agent that is or can be made responsive to an electric field, e.g., ionized, may be used in accordance with the methods of the invention. Non-limiting examples of agents that may be used in accordance with the methods and apparatus of the invention include naturally occurring and/or synthetic nucleic acids, peptides, peptide mimetics, polypeptides, antibodies, antigen-specific antibody fragments, and small molecules. Agents that may be used in accordance with the methods of the invention include therapeutics, investigationals and diagnostics.

The nucleic acids for use in accordance with the methods of the invention include, but are not limited to, DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions. In particular embodiments, the nucleic acid for use in accordance with the methods of the invention is a therapeutic nucleic acid as known in the art and/or described herein, e.g., an antisense nucleic acid, an siRNA, a short hairpin RNA, or an enzymatic nucleic acid.

The antibodies for use in accordance with the methods of the invention include, but are not limited to, monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site.

In certain embodiments, the agent for use in accordance with the methods of the invention is a neuroactive agent, modulating the activity of one or more types of CNS cells. For example, the methods of the invention provide for the management, treatment, or prevention of a CNS disease or disorder, or the management, treatment, prevention or amelioration of one or more symptoms of a CNS disease or disorder by, e.g., promoting the survival or death of a particular phenotype of a neuron or a particular region of CNS tissue, modulating synapse formation or activity (e.g., by the use of a neurotransmitter uptake inhibitor), modulating electrical activity of a neuron (e.g., by the use of calcium ion channel inhibitors), modifying the activity of a first neuron by effecting a response or activity in a second cell of the CNS, e.g., a microglial cell. Non-limiting examples of neurotransmitter uptake inhibitors that may be used in accordance with the methods of the invention to modulate the activity of CNS, e.g., neural tissue, include serotonin, dopamine and norepinephrine.

In one aspect, the invention also provides kits for the treatment of CNS disorders comprising the use of TCE, optionally in combination with CEDD, which kits comprise a delivery device useful for TCE or for combination TCE and CEDD, preferably a reflux-free cannula/catheter comprising a polarizable electrode, and one or more separately polarizable electrodes. The separately polarizable electrodes may be surface style electrodes that transmit the field through the surface of the skin (e.g., scalp) to the target tissue (e.g., brain) or may be electrodes designed for implantation in or remote to target tissues (e.g., the brain surface or within the CNS parenchyma), or combinations thereof.

5.1 Trans-Cerebral Electrophoresis

The invention provides for the generation of an electric field within or encompassing the target tissue to effect the trans-tissue electrophoresis and targeted delivery of a therapeutic, diagnostic, or investigational agent. Multiple methods exist for the generation of such an electric field in vivo, in particular, within the brain or parts of the CNS of a subject in need thereof. Examples of such methods include, but are not limited to, the use of external plates surrounding, but not touching, the target tissue and/or subject, surface electrode arrays, penetrating electrode arrays, and combinations of surface and penetrating electrode arrays. The invention can also be practiced with any electrode system suitable for propagating the electrical signals within or encompassing the targeted region of tissue. The specific characteristics of the electrode systems will determine if that type of electrode is suitable for use in a given application.

The most common use of electrode systems in the CNS in current clinical practice is the use of single, bipolar electrodes capable of stimulating or lesioning a target tissue. Because the target tissues of the CNS are comprised primarily of closely packed neural tissue, such electrodes are designed to affect a relatively small area immediately proximal to the electrode; stimulation or lesioning of larger areas would result in unknown and potentially undesirable side-effects. Accordingly, these electrodes are primarily designed as single lead bipolar or multichannel electrodes. In contrast, the apparatus and methods of the instant invention comprise an array of separately polarizable electrodes. In certain embodiments, the apparatus of the invention comprises an array of at least two separately polarizable electrodes, one of which is optionally housed in a cannula or catheter, e.g., an integrated TCE cannula as described herein, for application of an agent, e.g., a therapeutic or diagnostic agent. In another embodiment, the apparatus of the invention comprises an array of more than two separately polarizable electrodes (i.e., a plurality of electrodes), at least one of which is optionally housed in a cannula or catheter for application of the agent to be delivered, e.g., an integrated TCE cannula as described herein. In a specific embodiment, each electrode in the array of the apparatus of the invention is independent from the means of delivery of the therapeutic agent. In a specific example in accordance with this embodiment, the electrodes of the apparatus may be plates external to, but not touching, the subject and surrounding the target tissue.

The array of separately polarizable electrodes can be polarized by independent connection, for example, to a variable voltage power source, e.g., such as a battery, and activating the power source. Whether a specific polarizable electrode is charged negatively or positively will be a function of the location of agent application with respect to the location of the target tissue and the charge of the agent. The number, position, and charge of the polarizable electrodes can be determined by any method known in the art or described herein for estimation of agent response in vivo to a developed electric field, e.g., by use of computer-based three-dimensional simulation (e.g., finite element analysis software packages (e.g., COMSOL Multiphysics, (COMSOL, Inc., Burlington Mass.); FEMPRO (ALGOR, Inc., San Rafael Calif.)) and/or other methods known in the art (see, e.g., Lee et al., 2007, International Journal of Control, Automation, and Systems 5:337-342., encompassed by reference herein in its entirety).

When using simulation procedures, the target tissue location can be identified using any method known in the art, e.g., magnetic resonance imaging (“MRI”). The target location can then be simulated in three dimensional space using a computer based system and the effects of the electrical field and tissue composition on the charged agent can be simulated. The amount of the agent and the appropriate electrical field can then be determined to establish not only the desired concentration but also the residency time of the therapeutic agent within the target tissue.

In certain embodiments, the apparatus of the invention comprises surface-style electrodes, e.g., plates or meander-type electrodes (see e.g., U.S. Pat. No. 5,968,006, which is incorporated herein by reference in its entirety). Surface-style electrodes propagate the electric field through the surface of the skin and into the target tissue. In other embodiments, the apparatus of the invention comprises implantable, or penetrating, electrodes. Implantable electrodes useful for the generation of an electric field within the tissues of the CNS include, but are not limited to, electrodes designed to be inserted beneath the surface of the skin along the cranium, those designed to be inserted in the epidural space of the vertebral column or cranium, or those implanted along the surface of the brain, brainstem, or spinal cord. Penetrating electrodes are conductive elements whose size and shape are sufficient to enable insertion through the matter covering a tissue of interest or through the tissue of interest itself. Penetrating electrodes are well known in the art, and have, for example, been used to treat chronic pain, symptoms of Parkinson's disease, epilepsy, hearing disorders, depression, obsessive/compulsive disorder, and muscle disorders.

Electrode design is a critical component of TCE. Electrode parameters include diameter, conducting surface geometry, length, conductivity and materials. In certain embodiments, the electrodes are hollow, allowing for the administration of an agent, e.g., diagnostic, therapeutic or anesthetic agent. In other embodiments, the electrodes are coated with anesthetics and/or lubricious agents for pain mitigation and ease of insertion. The design or selection of the electrode is determined by several treatment factors, including properties of the target tissue, tissue volume to be treated, and charge injection/current densities at the electrode-tissue interface. The inter-electrode spacing and penetration depth define the shape of the electric field, and thus the volume of tissue to be treated. In certain embodiments the spatial arrangement of the electrodes surrounding or within the target tissue may be based on computer simulation of the electric field and the agent response thereto. Such simulations may be developed by any method known in the art, e.g., using finite element analysis software such as, but not limited to, COMSOL Multiphysics (COMSOL, Inc., Burlington Mass.); FEMPRO (ALGOR, Inc., San Rafael Calif.) and/or IPlan Software (BrainLab, Inc., Munich Germany).

The electric field generated between or among the two or more electrodes creates an EMF that moves the charged agent in a controlled fashion so as to achieve the desired agent concentration/distribution for a specified time within the target tissue, thereby generating the desired effect. Because TCE is used to direct or regulate the movement of the agent to or within the target tissue, the delivery location of the agent need not be directly to the target location, but can be at a remote site. In such embodiments, to effectuate the desired movement of the agent within the tissue, the electrical field is preferably adjustable/changeable. Moreover, the polarity of two or more of the polarizable electrodes can be switched to manipulate the direction of the movement of the charged agent within the tissue. The strength of the electrical field can also be adjusted to control the rate of movement of the charged therapeutic agent to and within the tissue.

It is preferred that the electrodes have a sufficiently inert surface material that is electrochemically stable and will not exhibit substantial oxidation-reduction reactions within the interstitial environment when exposed to the electric current. Non-limiting examples of such surfaces include gold, nickel, titanium, titanium nitride, platinum, platinum-iridium, iridium, iridium-oxide, silver, silver-plated copper, silver tungsten, silver cadmium-oxide, silver tin-oxide, indium-tin-oxide, and tin-oxide. Depending upon the material chosen, it may be desirable for cost and structural reasons to deposit these inert metals to the surface of a base metal. Appropriate base metals include, but are not limited to titanium, tungsten and stainless steel. As known in the art, the level of charge injection and irreversible oxidation-reduction reactions are parameters to be considered when choosing a sufficiently inert material and deposition thickness.

In certain embodiments, dielectric coatings are deposited on the surface of the electrode to avoid generation of non-homogeneous electrical fields. Such dialectic coatings are typically deposited at the level of tenths to hundreds of microns thick Non-limiting examples of suitable dielectric coatings include polytetrafluoroethylene (PTFE), parylene, and silicon carbide. In certain embodiments, the electrode is covered in a biocompatible insulating material except for a small region to allow a contact through which charge may flow.

The electric field encompassed by the invention is preferably less than that required to stimulate the excitable tissue(s) of the CNS. Stimulation or lesioning methods generally require high frequency AC current (up to approximately 200 μamps). In contrast, the instant invention comprises methods using low frequency AC, low frequency DC pulses or DC current. Without being bound by any particular mechanism of action, it is believed that the DC current or low frequency pulses establish an EMF sufficient to effect transfer of charged therapeutic agents through the target tissue. To avoid damage to the CNS during TCE, the invention uses a current of low amperage to establish the electric field. In certain embodiments, the electric current is no greater than 10 mA. In other embodiments, the current is no greater than 8 mA, 6 mA, 4 mA, 2 mA, 100 μA, 75 μA, 50 μA, 25 μA, 15 μA, 10 μA, 8 μA, 6 μA, 4 μA, 2 μA or 1 μA. Because of the low amperage, it is envisioned that, for certain embodiments, the mobility effects of the methods of the invention require the subject to undergo prolonged TCE. In certain embodiments, the methods of the invention encompass one or more round of TCE of about 1 h, 2 h, 4 h, 6 h, 8 h, 10 h, 15 h, 20 h, 24 h, 1.5 days, 2 days, 5 days, 1 week or 2 weeks, duration. However, in certain embodiments involving full subcutaneous implantation of the apparatus, TCE may be implemented as a repeated or continuous treatment for months or years. In certain embodiments, TCE according to the methods of the invention may be effected by the use of electrode plates external to, but not touching, the patient and surrounding the target tissue. Alternatively, for TCE according to the methods of the invention the apparatus of the invention may be designed for acute implantation. In other embodiments of the invention, TCE is chronically applied to the target tissue, e.g., with chronic administration of therapeutic agents, and, accordingly, the apparatus of the invention is designed for permanent or chronic implantation.

In certain embodiments, the electric field is generated by plates external to, but not touching, the subject and surrounding target tissue. In specific examples in accordance with this embodiment, the electric potential between the two external plates is from 1-100 V, 1-80 V, 1-60 V, 1-40 V, 1-20 V, 1-10 V, 1-5 V, 5-600 V, 5-500 V, 5-400V, 5-300 V, 5-200 V, 5-100 V, 100-600 V, 100-500 V, 100-400 V, 100-300 V, or from 100-200 V. In specific embodiments, the electric potential between the two plates is 5 V, 8 V, 10 V, 15 V, 20 V, 25 V, 50 V, 100 V, 150 V, 200 V, 250 V, 300 V, 350 V, 400 V, 450 V, 500 V, 550 V or 600 V.

In other embodiments, the electric field is generated by two or more implantable electrodes, or a combination of two or more implantable and surface electrodes. In specific examples in accordance with this embodiment, the electric potential between the reference electrode any other of the array may be from 2-20 V, 5-20 V, 10-20 V, 10-18 V, 10-16 V, 10-14 V or from 10-12 V. In specific embodiments, the electric potential between the reference electrode and any other of the array is 2 V, 4 V, 6 V, 8 V, 10 V, 12 V, 14 V, 16 V, 18 V, or 20 V. In embodiments of the invention comprising the use of more than two electrodes, the electric potential between any two electrodes within the array may be the same or different from that between any other two. A varied gradient within the array may be useful, e.g., for the creation of a concentration gradient of the applied agent within the developed electric field.

The power source to the electrodes is capable of delivering alternating current (AC) or direct current (DC). The current may be delivered by an andoal and a cathodal segment. In certain embodiments, the current is pulsed. In the AC embodiment, the pulse frequency is generally low, about 10 Hz or less. In specific examples in accordance with this embodiment, the pulse frequency is 5 Hz or less, 2 Hz or less, 1 Hz or less, 0.5 Hz or less, 0.1 Hz or less, 0.05 Hz or less, 0.01 Hz or less, 0.005 Hz or less, 0.001 Hz or less, 0.0005 Hz or less, or 0.0001 Hz or less. For non-pulsed DC current, the pulse frequency is 0. Pulse width may be varied to provide optimum dispersion of the administered agent. In certain embodiments, the pulse width, or signal duration, is from 1 microsecond (“μs”) to 5 seconds (“s”), 1 μs to 2 s, 1 μs to 1 s, 1 μs to 500 milliseconds (“ms”), 1 μs to 200 ms, 1 μs to 100 ms, 1 μs to 50 ms, 1 μs to 20 ms, 1 μs to 10 ms, 1 μs to 1 ms, 1 μs to 500 μs, 1 μs to 100 μs, 1 μs to 50 μs, 1 μs to 10 μs, 1 ms to 200 ms, 1 ms to 100 ms, 1 ms to 5 ms, or 1 ms to 20 ms. In other embodiments, the pulse width, or signal duration, is from 1 ms to 5 s, 1 ms to 2 s, 1 ms to 1 s, 1 to 500 ms, 1 to 200 ms, 1 to 100 ms, 1 to 50 ms, 1 to 20 ms, 1 to 10 ms, 10 to 200 ms, 10 to 100 ms, 10 to 50 ms, or 10 to 20 ms. The pulse widths of the anodal and cathodal segments are either symmetric or can be asymmetric. It is believed that an asymmetric wave offer reduced pH changes at the electrode surface. Train length may be from hours to days, dependent on pulse width and frequency. The invention contemplates any pulse shape, or current waveform, including bipolar, monopolar, capacitive discharge, square, sawtooth, or any combination of the foregoing. In certain embodiments, the pulse shape is a square wave. It is believed that square waves may offer further improved agent dispersion due to the impulse nature of the developed EMF.

In certain embodiments, the invention encompasses the monitoring of one or more of the electrodes in the array, in particular the resistance of the one or more electrodes, such that the charge to the electrode can be varied and/or controlled to generate the desired field within the target tissue. In other embodiments, the one or more electrodes comprise a thermocouple to monitor the temperature of the tissue surrounding the electrode. The temperature at the electrode site may be monitored for levels of heat that may lead to tissue damage. Safety devices may be included as part of the apparatus of the invention to automatically stop TCE or switch off the apparatus when a set temperature is reached or exceeded. The safety temperature may vary depending on the length of time the tissue is to be exposed to the electric current, as high temperatures may be tolerated for brief periods, but, generally, the safety temperature is not greater than 40° C. In other embodiments, the one or more electrodes comprise a microtube or capillary through which cooled fluid (for example, saline) may be pumped to maintain the temperature of the electrode and/or surrounding tissue at or below the safety temperature. In preferred embodiments, the microtube or capillary forms a fluid path within the interior of the one or more electrodes such that the path does not disrupt the conductive surface of the electrode in contact with the tissue of the subject. In such embodiments, the microtube or capillary within the electrode has an inflow and outflow comprising suitable connectors for fluid connection to a reservoir or other source of cooling fluid to form a cooling system. The apparatus of the invention may comprise one or more pumps, valves or flow initiators/controllers in fluid connection with a cooling apparatus and the microtubes/capillaries of the one or more electrodes to provide a flow of fluid through the microtubes/capillaries to maintain or reduce the temperature of the electrode or tissue. In certain embodiments each electrode of the electrode array comprises a microtube/capillary as described herein and is fluid connection with cooling system; in other embodiments, only one, a minority, about half or more than half but not all of the electrodes of the array comprise a microtube/capillary as described herein in fluid connection with the cooling system. The cooling system may receive input from the thermocouples of the electrodes as described herein to automatically regulate the temperature of the one or more electrodes and/or surrounding tissue. The flow of fluid through the microtube/capillaries of the one or more electrodes of the electrode array need not be continuous, but may be regulated by manual or processor control. In preferred embodiments the fluid path through the microtubes/capillaries and other components of the cooling system is closed such that the cooling fluid does not come into contact with the tissue of the subject. Because the cooling fluid does not contact the subject, any suitable cooling fluid known in the art may be used, but is preferably biocompatible and/or non-toxic.

To ensure that the proper concentration of the administered agent is reaching the target location, the concentration of the agent in the subject tissue can be measured at certain points in/around the target location. The concentration of the charged agent can be measured using any technique known in the art and/or described herein, e.g., a microdialysis technique. The measured concentration can be compared with a desired concentration. If the measured concentration of the agent within the target location is not approximately equal to the desired concentration, the delivery of the agent to the target location can be adjusted accordingly. For example, the strength of the electrical field, an alteration of the polarity of one or more of the polarizable electrodes, and/or an adjustment to the rate of application/delivery of the administered agent to the tissue may each be independently or concomitantly adjusted to obtain the desired concentration at the target location.

5.2 Convection Enhanced Drug Delivery (“CEDD”)

In certain embodiments, TCE is combined with Convection Enhanced Drug Delivery (“CEDD”) techniques. CEDD, also known as high-flow interstitial infusion, is a technique well known in the art, and involves the application of an agent under pressure to a tissue structure. The pressure generated by the delivery system is believed to provide convection assisted agent dispersion within the target tissue. CEDD generally requires positioning the tip of one or more infusion catheters or cannulas (e.g., an integrated TCE cannula as described herein), preferably a reflux-free catheter or cannula, within a tissue structure and provision of a solution comprising an agent to be administered through the catheter/cannula while maintaining a pressure gradient from the tip of the catheter during the infusion. Most commonly, the pressure gradient is created by connecting the one or more infusion catheters/cannulas to a pump after positioning in the tissue situs as is well known in the art (see, e.g., Saito et al., 2005, Exp Neurol, 196:381-389; Krauze et al., 2005, Exp Neurol, 196:104-111; Krauze et al., 2005, Brain Res Brain Res Protocol., 16:20-26; Noble et al., 2006, Cancer Res 66:2801-2806; Saito et al., 2006, J Neurosci Methods 154:225-232; Hadaczek et al., 2006, Hum Gene Ther 17:291-302; Hadaczek et al., 2006, Mol Ther 14:69-78, U.S. Patent Application Publication No. 2006/0073101; and U.S. Pat. No. 5,720,720, each of which is incorporated herein by reference in its entirety). The pumps of the apparatus of the invention may be implantable pumps (including but not limited to active and passive drug delivery systems (see, e.g., U.S. Pat. Nos. 7,351,239; 4,629,147; 4,013,074 each of which is hereby incorporated by reference in its entirety)) or external pumps (e.g., roller pumps or syringe pumps) and may deliver one or more separate agents to one or more delivery sites within the tissues of the subject. In specific embodiments, the apparatus of the invention comprises a CEDD-compatible reflux-free step design cannula (see, e.g., Krauze et al., 2005, J Neurosurg 103:923-9, and U.S. Patent Application Publication Nos. US 2006/0135945 and US 2007/0088295, each of which is incorporated herein by reference in its entirety).

In specific embodiments, the distal end of the one or more infusion catheters/cannulas and/or integrated TCE cannulas is a needle-like assembly, such as a stainless steel or stiff polymer tube having one or more elongated ports to release the therapeutic agent in a discrete location or over an extended site. The more proximal portions of the infusion catheter/cannula may be formed of one or more segments of any biocompatible material of a suitable stiffness to dependably transmit microdose or microflow volumes of compositions comprising the therapeutic agent from the pump through the catheter/cannula, without loss of pressure. In certain embodiments, the invention comprises the use of more that one infusion catheter/cannula for application of the therapeutic agent at more than one tissue situs. In other embodiments, the infusion catheter/cannula has one or more sensors to monitor apparatus performance and/or method parameters, e.g., drug concentration at the site of application, tissue condition (e.g., temperature). In still other embodiments, the one or more infusion cannulas/catheters of the invention are primed with a pharmaceutically acceptable carrier and/or a pharmaceutical composition prior to implantation or insertion into the tissue situs.

The invention encompasses CEDD at any suitable flow rate such that the intracranial pressure is not increased to levels injurious to tissues of the brain. In certain embodiments, the infusion flow rate is from 0.1-1000 μL/hr, 0.1-900 μL/hr, 0.1-800 μL/hr, 0.1-700 μL/hr, 0.1-600 μL/hr, 0.1-500 μL/hr, 0.1-400 μL/hr, 0.1-300 μL/hr, 0.1-200 μL/hr, 0.1-100 μL/hr, 0.1-80 μL/hr, 0.1-70 μL/hr, 0.1-60 μL/hr, 0.1-50 μL/hr, 0.1-40 μL/hr, 0.1-30 μL/hr, 0.1-25 μL/hr, 0.2-20 μL/hr, 0.1-15 μL/hr, 0.1-10 μL/hr, 0.1-5 μL/hr, 0.1-2 μL/hr, 0.1-1 μL/hr, 0.1-0.8 μL/hr, 0.1-0.6 μL/hr, 0.1-0.5 μL/hr, 0.1-0.4 μL/hr, 0.1-0.3 μL/hr, 0.1-0.2 μL/hr, 0.1-25 μL/min, 0.5-20 μL/min, 1-15 μL/min, 1-10 μL/min, 1-5 μL/min, or 1-2 μL/min. In specific embodiments, infusion flow rate is about 0.1 μl/hr or less, about 0.5 μL/hr or less, about 0.7 μL/hr or less, about 1 μL/hr or less, 0.1 μl/min or less, 0.5 μL/min or less, about 0.7 μL/min or less, about 1 μL/min or less, about 1.2 μL/min or less, about 1.5 μL/min or less, about 1.7 μL/min or less, about 2 μL/min or less, about 2.2 μL/min or less, about 2.5 μL/min or less, about 2.7 μL/min or less, about 3 μL/min or less, about 5 μL/min or less, about 7 μL/min or less, about 10 μL/min or less, about 12 μL/min or less or about 15 μL/min or less. In preferred embodiments, the infusion flow rate is 5 μl/min or less. In other embodiments, the invention provides for CEDD comprising incremental increases in flow rate, referred to as “stepping”, during delivery.

The distal end of the catheter/cannula is implanted providing a fixed site of agent administration, and, in certain embodiments, extends such that one or more ports of the catheter open in or near the target site, which may, for example, be a tumor site, a nerve, a lesion or other targeted region of affected brain or other CNS tissue. Because TCE used in conjunction with CEDD will effect dispersion of the administered agent, there is not a requirement to have the one or more ports of the catheter/cannula open directly in the target tissue. Thus, in certain embodiments, the invention encompasses insertion of the catheter/cannula into a non-target tissue situs and use of TCE to ensure contact between the administered agent and the target tissue. The freedom of administration site may allow tissues to be targeted according to the methods of the invention that are otherwise unsuited for treatment using standard CEDD.

In certain embodiments, the distal end of the catheter/cannula is stereotactically implanted into brain tissue through a cranial hole to deliver the agent into the parenchymal spaces. The remaining components of the CEDD system, e.g., infusion pump and power supply, need not be near the one or more infusion catheters but may be connected via appropriate electrical connections and tubing. For long term infusions according to the methods of the invention, the invention encompasses chronic implantation of the infusion catheter. In such embodiments, the remaining components of the CEDD system and/or TCD apparatus as described herein may also be implanted subdermally. For example, in certain embodiments, the fluid supply to the inlet of the infusion pump is an implanted reservoir or other supply. In other embodiments, the fluid reservoir is implanted subdermally and possesses a cover or septum formed of a self-sealing polymer. Such reservoirs are refillable through the patient's skin by piercing the septum with a syringe to deliver a refill volume of the fluid comprising the therapeutic agent. In still other embodiments, the reservoir is a pressurized assembly, such as a pressure-driven bellows, in which case the infusion pump assembly may be implemented by simply providing one or more valves, restrictors or other elements that regulate the time and/or the rate at which fluid is allowed to pass from the reservoir. In still other embodiments, the infusion pump is an electrically powered assembly, having a power source and a controller.

In specific embodiments, the invention provides for one or more chambers in the pump assembly that contains one or more concentrated agents to be administered, which the assembly combines with one or more carrier fluids. A mixing chamber may be provided to allow mixing of the one or more agents and one or more carriers before they are pumped to the tissue site. This is especially advantageous, for example, in multidrug regimens in which several incompatible or mutually unstable drugs are to be delivered at once, or in which concentration must be closely controlled. In other embodiments, dispersion of the administered agent is further augmented by the use of a facilitating agent, such as low molecular weight heparin.

5.3 TCE Apparatus

In exemplary embodiments, the invention provides an integrated TCE cannula/catheter that functions as both a cannula for infusion of an agent into a tissue situs and at least one monopolar electrode of a monopolar electrode array. The integrated TCE cannula may function as part of a TCE apparatus comprising two subsystems according to the methods of the invention: the first, a CEDD infusion system, e.g., including a programmable infusion pump and one or more infusion catheters/cannulas, through which an agent may be delivered under pressure into a targeted tissue situs and; the second, an array of two or more electrodes connected to a current source with which the trans-tissue, e.g., trans-cerebral, electric potential gradient is created, one of electrodes is, optionally, the at least one monopolar electrode of the integrated TCE cannula. In alternate embodiments, the invention provides for a method encompassing the use of one or more infusion catheters to administer the agent and two or more electrodes to establish an electric field within the target tissue. In specific embodiments, the methods of the invention provide for the use of separable systems, e.g., wherein the one or more infusion catheters are not combined with one or more electrodes and/or are not connected to a current source. In other embodiments, the invention provides one or more integrated infusion catheters comprising one or more polarizable electrodes. In specific embodiments, the integrated TCE catheter of the invention is surgically implanted into the desired CNS site (e.g., parenchymal site) and the remaining one or more electrodes are placed along the surface of the skin covering or surrounding the target tissue site and/or are implanted into the CNS tissue. For example, in those embodiments wherein the CNS tissue is the brain, the remaining one or more electrodes may be placed along the brain surface, within the brain parenchyma, within the epidural space, within the skull, under the scalp, or along the surface of the scalp. The infusion catheter and electrode wires may be tunneled subcutaneously and connected, respectively, to a pump and current source, optionally a pulse generator, which may either be external to the body or implanted subcutaneously. In specific embodiments, both the infusion pump and the implantable current source may be programmed transcutaneously.

In certain embodiments the invention provides for a processor which is in communication with the electrodes of the array and can send and receive signals to other external components of the system, directing the activity of the other components, e.g., the infusion pump and the power source, in response to the electrode signals. The electrode signals may include determinations of electrode resistance or, for electrodes comprising a thermocouple, temperature. In certain embodiments, the processor can be used independently or concomitantly to regulate the flow of the therapeutic agent from the infusion pump, to regulate the intensity and shape of the electric field within the target tissues by regulating the output of the power source (e.g., current or voltage generator) or to selectively power only a subset of the electrodes in the array. In certain embodiments, the connection between one or more electrodes and the processor comprises resistors for modulating the impedance of the electrode, allowing electrodes with increased impedance to function as a recording electrode, i.e., used to provide information to the processor. The connection between a recording electrode and the processor may also include one or more preamplifiers for amplifying signal received from the recording electrode.

In certain embodiments, the apparatus of the invention comprises one or more thermocouples. In specific embodiments, the thermocouple is implanted in the target tissue and is separate from any other implantable or surface component of the apparatus of the invention, e.g., an electrode, infusion catheter/cannula. In other embodiments, the thermocouple is integrated into one or more implantable or surface components of the apparatus, e.g., electrode, infusion catheter/cannula. The thermocouple monitors the temperature of the tissue surrounding or in contact with the apparatus component, including, for example, the tissue of the target area, and may, optionally, be connected to a processor regulating the TCE apparatus of the invention. The output of the thermocouple may be used by the human operator or the control processor to adjust apparatus parameters, e.g., current output, voltage, so as to avoid tissue damage from exposure to the electric current.

The invention provides for the use of biocompatible materials to form the components of the integrated TCE cannula and/or apparatus, which components are interconnected by one or more leads. The leads may extend from components such as one or more electrodes to, e.g., a power source and/or a processor for regulating the function of the apparatus. Where in contact with the tissue of the subject, leads are placed within a biocompatible, sterilizable, flexible or semi-flexible sheath. As used herein, the term “source devices for the electrode array” describes a device comprising a battery or power source to power the electrode array, a pump or delivery device for agent administration, and, optionally, a processor for providing instructions to the apparatus. In certain embodiments, the source devices are implantable and/or portable and/or self-regulating. In other embodiments, source devices are extracorporeal device and may be controlled by the patient and/or a health care worker.

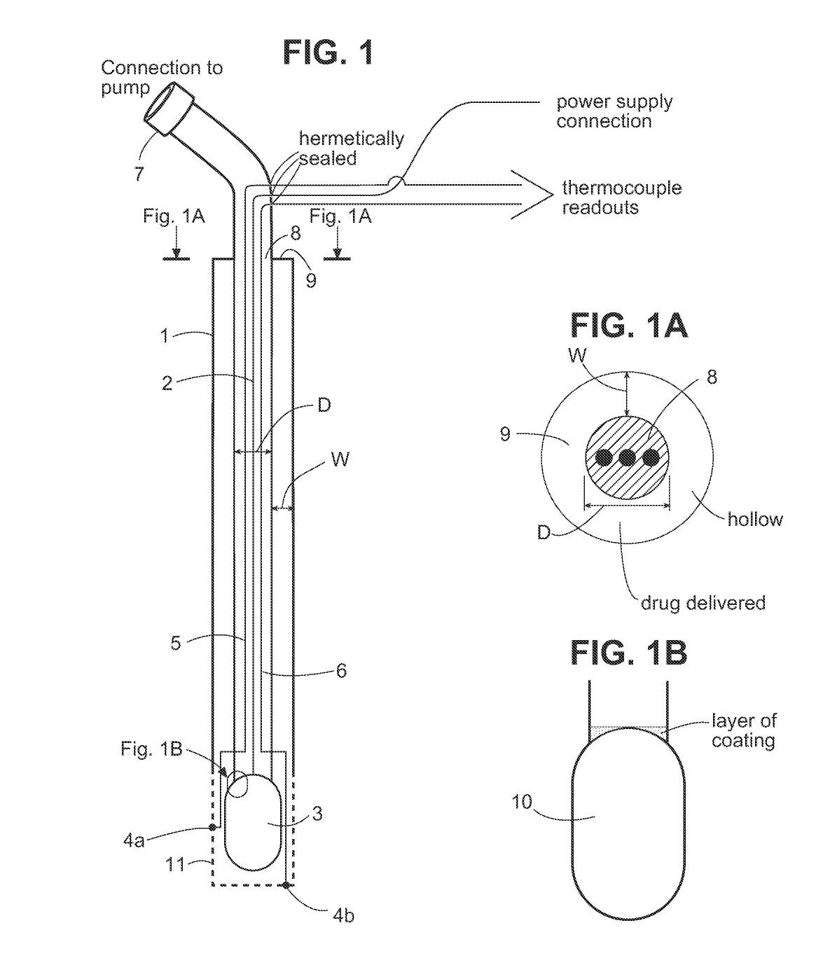

FIGS. 1, 1A and 1B schematically illustrate the integrated TCE cannula, which cannula comprises an integrated infusion catheter/cannula and electrode. Throughout the figures, like elements are identified by like numbers. The integrated TCE cannula is a hollow tube with an outer wall (1) formed from any biocompatible, sterilizable material of suitable stiffness to allow target tissue implantation and delivery of microflows of solutions comprising one or more therapeutic agents to be delivered under pressure. The integrated TCE cannula comprises a solid inner core (8) containing one or more electrical leads (2, 5, 6) that connect electrical components at the base or side of the cannula to source devices of the apparatus, e.g., a power source and/or processor. For example, the solid inner core (8) may comprise a plurality of wires or leads embedded in a solid plastic (e.g., polyurethane) material. In certain embodiments, the electrical leads are hermetically sealed. Each lead is separated from the other by a region of insulating material such that there is no electrical cross-talk between the leads. For example, as shown in FIG. 1B, each lead (10) may comprises a thin layer of insulating coating, e.g., thin plastic coating, having a thickness of about 0.01 to about 0.5 mm, preferably, the coating has a thickness of about 0.1 mm.

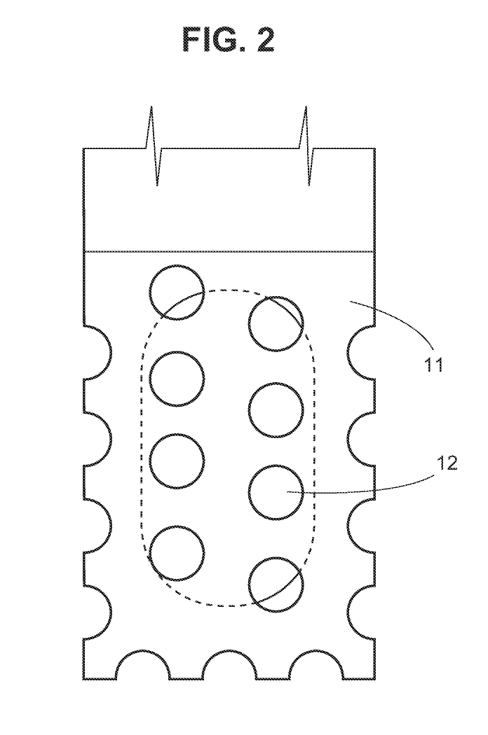

The central core maybe formed completely from the insulating material or may be formed from the material of the cannula and filled with the insulating material. In some embodiments, the solid inner, core (8) may have a diameter (D) of any suitable size, preferably from about 0.1 to about 10 mm, about 0.5 to about 5 mm, about 0.5 to about 1 mm or about 0.1 mm. Insulating materials are any materials having a dielectric constant greater than that of the lead metal. Non-limiting examples of insulating materials include glass fiber, silicon elastomers, TEFLON® (PTFE), plastics, including, but not limited to, polyurethane, and like materials having high dielectric constants. Typically, the entire lead is covered by insulating material except for region(s) at the tip (e.g., of about 2 to 5 μm) where “open contacts” or surfaces through which electrical current can pass are necessary, e.g., as in a thermocouple. The solid inner core creates a hollow space within the integrated TCE cannula (9) to allow flow of a solution through the cannula. In some embodiments, the solid inner core (8) may be concentric with the integrated TCE cannula (9). More specifically, a distance (W) between a radius of the solid inner core (8) and the integrated TCE cannula (9) may be of any suitable size, preferably from about 0.1 to about 10 mm, about 0.5 to about 5 mm about 0.8 to about 3 mm, about 1 to about 2 mm, or about 2 mm. The proximal end of the cannula is connected to an infusion pump to provide the solution comprising the therapeutic agent at a given flow rate (7). The distal tip of the outer wall of the integrated TCE cannula comprises one or more ports (i.e., holes)(12) through which the solution may exit the flow space (9) and flow into the tissue situs (see, FIG. 2).

In one exemplary embodiment, as shown in FIG. 2, the distal end of the cannula comprises a porous outer sheath (11), comprising a plurality of pores (12). The sheath (11) may be formed from any suitable bio-inactive plastic material, preferably a biocompatible polyurethane. The pores (12) may serve as ports to allow flow of fluids and drugs therethrough and into the tissue. The pores (12) may have any suitable shapes or sizes. For example, the pores (12) may have an irregular, circular or oval shape. In some embodiments, the pores (12) may have a diameter or largest transverse of about 0.01 mm to about 0.5 mm. In certain embodiments, the pores (12) may be variable in size. In particular, the pores (12) may be uniformly distributed throughout the sheath (11). In some specific embodiments, the pores (12) may occupy at least 10%, 25%, 30%, 40%, or 50% of the overall area of the sheath (11). In a particularly preferred embodiments, the pores (12) may be variable in size, uniformly distributed throughout the sheath (11) and occupy at about 50% of the overall area of the sheath (11).

In specific embodiments, the entire cannula is formed from an insulating material, e.g., polyurethane, and the ports or open contact areas at the distal end of the cannula (11, 12) allow electric current to flow from the electrode (3) into the tissue. The integrated TCE cannula comprises one or more polarizable electrodes (3). For example, the electrode (3) may comprise a Platinum/Iridium (Pt/Ir) electrode. The surface area of the electrode in electrical contact with the tissue of the subject is preferably sufficiently large to avoid high charge densities within the tissue. The one or more electrodes may be of any size or shape suitable for the generation of an electric field within the target tissue of sufficient strength to provide an EMF capable of dispersing the therapeutic agent through the target tissue while avoiding tissue damage from the presence of an electric current. In some embodiments, the electrode (3) may have a smooth or uniformly even surface to minimize areas for charge buildup on the surface. In certain embodiments, the electrode is an oblate spheroid to minimize variable charge densities over the surface of the electrode (which also provides a more uniform electric field within the tissue) with a longer axis of about 1 to about 10 mm, including axis of about 1 mm, 2 mm, 3 mm, 4 mm, 5 mm, 6 mm, 7 mm, 8 mm, 9 mm, or 10 mm; and the shorter axis of about 0.3 to 3 mm, including axis of about 0.3 mm, 0.6 mm, 0.9 mm, 1.2 mm, 1.5 mm, 1.8 mm, 2.1 mm, 2.4 mm, 2.7 mm or 3 mm. Other sizes and shapes, with either larger or smaller may also be used in accordance with the methods of the invention.

In other embodiments, one or more of the electrodes in the array is a surface electrode. The surface electrode may be of any shape suitable for the generation of an electric field within the target tissue of sufficient strength to provide an EMF capable of dispersing the administered agent through the target tissue while avoiding tissue damage from the presence of an electric current. In specific embodiments, the surface electrode is a disc of diameter about 0.5 to about 5 cm, including 0.5 cm, 1 cm, 1.5 cm, 2 cm, 2.5 cm, 3 cm, 3.5 cm, 4 cm or 5 cm. In certain embodiments, the integrated TCE cannula comprises one or more thermocouples that may be used to determine the temperature of the tissue at one or more contact points with the cannula (e.g., on the side (4a) or distal end (4b) of the cannula). In some embodiments, the thermocouples (4a and 4b) may be embedded into a porous outer sheath serving as open contact areas at the distal end of the cannula (11). More particularly, the thermocouples (4a and 4b) may also be coated with a thin layer of insulating material, such as plastic.

The integrated TCE cannula is configured to treat a target tissue in a patient's body, such as CNS tissue (including, but not limited to, the brain). In certain embodiments, one or more integrated TCE cannulas are implanted such that the fluid comprising the agent exits through one or more openings at the distal ends of the one or more cannulas at or remote to the target tissue site. The combination CEDD and TCE effected by the apparatus and methods of the invention as described herein effect a permeation of the one or more administered agents through targeted tissue to contact and effectively treat the targeted region(s). Thus, the region of effective tissue treatment is defined by both the cannula placement and the parameters of the electric field. The one or more cannulas may be placed at or near a brain tumor or diseased region of the brain, at or near a tumor to deliver a chemotherapeutic agent, at or near a nerve location to treat chronic pain, or at another suitable local site. In certain embodiments, one or more implantable components of the apparatus of the invention, e.g., one or more electrodes, one or more infusion catheter/cannulas, an integrated TCE cannula, may be implanted using image-guided, electrophysiologically-guided or stereotactic techniques to ensure correct spatial positioning of the one or more components.

Electrical continuity is necessary between the two or more polarizable electrodes and the electrical signal generator/power source. Therefore, in embodiments where the electrode array is detachable, a continuous and reliable electrical connection should be readily achieved between the two or more electrodes within the array and apparatus source devices, e.g., the power generators and optional regulators.

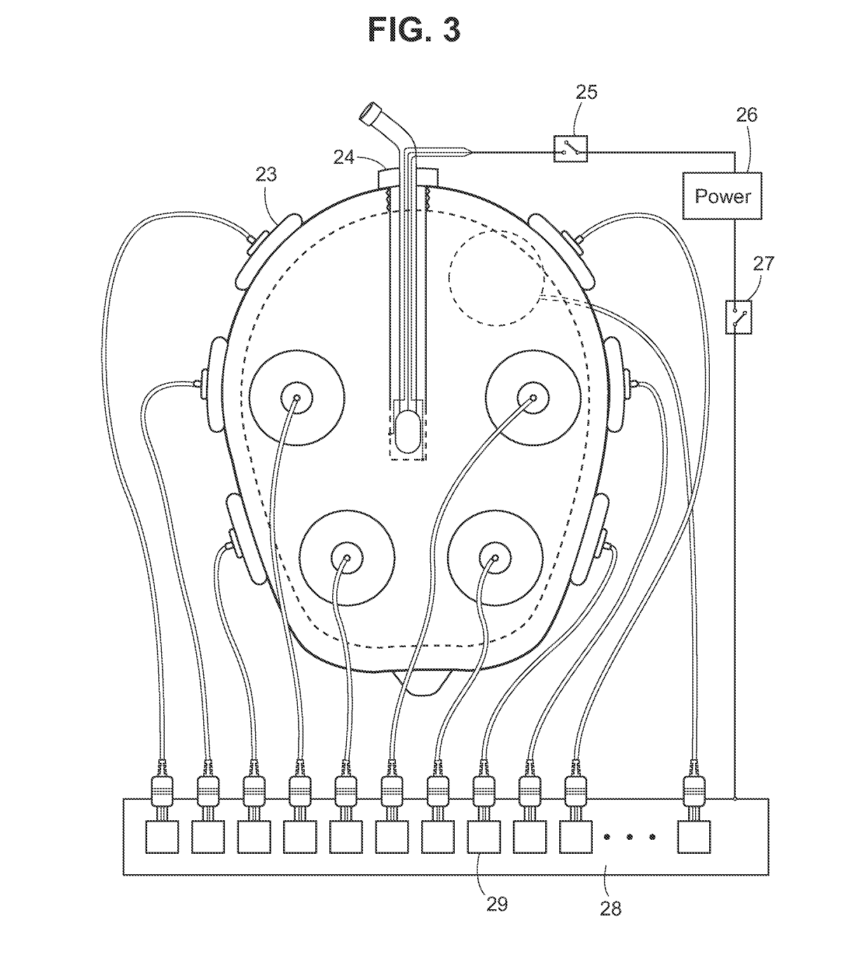

The invention also includes an electrical signal generator in conductive communication with the electrodes and/or integrated TCE cannula. The nature of the electrical signal generator will depend on the desired application. In some embodiments, the electrical signal generator is located within or physically connected to, by means other than electrical leads, the integrated TCE cannula. In the case of external electrical signal generator, a cable between the generator and the two or more electrodes of the apparatus is provided with a suitable connector to the two or more electrodes in a manner to minimize interference with operator use. In certain embodiments, the apparatus comprises a plurality of electrodes. In such embodiments, the plurality of electrodes may be organized into sets of channels, each set comprising at least two, and preferably, at least four channels for connection to the apparatus source devices. FIG. 3 schematically illustrates exemplary components of one embodiment of the apparatus of the invention, which embodiment comprises a combination of surface and implantable electrodes. The plurality of electrodes represented in FIG. 3 comprises one single implanted electrode secured by a locking cap on the skull of the subject, e.g., an integrated TCE cannula as described herein, (24) and a series of surface electrodes (23). The electrode array is attached to a processor or control mechanism (28) and power source (26 and 27). In certain embodiments, each electrode has an independent circuit (29) within the processor or control mechanism (28) to monitor the impedance of each electrode, to monitor the parameters of the developed electric field and to provide safety switches for cutting power to the electrode should any parameter exceed safety levels.

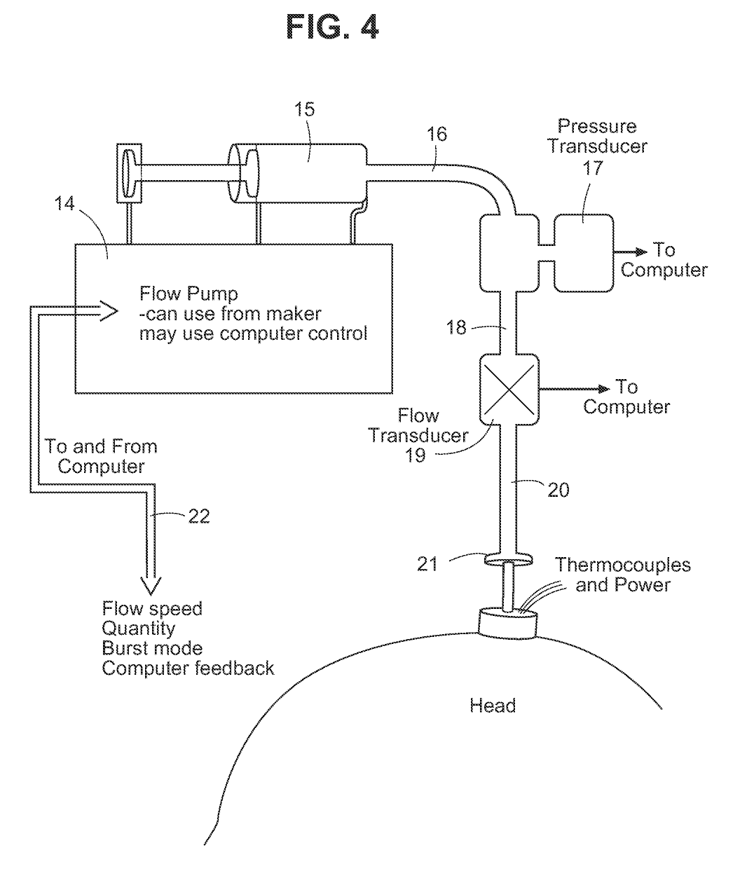

In specific embodiments, the invention provides for one or more infusion reservoirs or sources of fluid(s) connected through a pump, valve or flow initiator/controller. The pump, valve or flow regulator is fluidly connected to the infusion catheter of the invention, e.g., an integrated TCE cannula, to provide a solution comprising one or more agents through the catheter into a tissue situs. FIG. 4 schematically illustrates exemplary components of one embodiment of the invention, which embodiment comprises an infusion reservoir (15) connected to a pump or flow initiator/controller (14). The infusion reservoir may be one or more containers for holding fluid to be introduced to one or more tissue sites, which containers are capable of maintaining the sterility of the fluid. In specific embodiments, the one or more fluid reservoirs are one or more syringes. The pump or flow initiator/controller (14) can independently control the one or more fluid reservoirs and may further comprise programmable safety switches to halt flow should the pressure in any line from the pump become too great. In certain embodiments, the pump or flow initiator/controller is operated by a health care worker or the subject. In other embodiments, the pump or flow initiator/controller is operated remotely or by a processor that has been programmed to regulate pump functions and safety. The output of the pump is fluidly connected to the infusion catheter, e.g., the integrated TCE cannula (16, 18, 20). The fluid connection may be made of any suitable sterilizable material capable of maintaining both the pressure generated by the pump and the sterility of the fluid from the pump. In certain embodiments, the fluid connection may also comprise additional components for monitoring pump performance such as a pressure or flow transducer (17 and 19, respectively). The fluid reservoirs need not necessarily contain the administered agent (e.g., therapeutic or diagnostic agent), but, in certain embodiments, contain only one or more pharmaceutically acceptable carriers. In such embodiments, the one or more agents may be introduced at any point in the fluid connection between the pump or flow initiator/controller and the infusion catheter/cannula (21), e.g., introduced via a standard IV port in the fluid connection. Such embodiments are particularly useful wherein two or more agents are to be used that have varying stability, handling and/or storage requirements. The precise pump structure may be flexibly implemented with any suitable structure as known in the art, either with an electromechanically-actuated peristaltic or displacement pumping mechanisms, or with a pressurized reservoir or osmotically-driven source connected to a control valve or restrictor assembly to regulate the provision of fluid into the fluid delivery path. In either case, whether powered by pressure or electromechanically, the infusion pump assembly produces an accurately administered and sustainable flow of a total volume of fluid at a suitable infusion flow rate. In accordance with one aspect of the invention, the pump or flow initiator/controller provides a flow of fluid through a release device that is effective to increase the pressure locally at the region of the infusion catheter/cannula distal outlet ports, where the catheter/cannula is implanted in tissue, creating a pressure gradient that drives bulk transport of the drug into the target tissue site. For a typical implanted brain catheter/cannula delivery route, such pressure gradients are normally achieved with a flow rate of about 0.5 to about 20.0 μl/min. The fluid flow may be set and the pump assembly actuated based upon modeled properties such as histological tissue traits, therapeutic agent and carrier viscosity, infusion catheter and port dimensions and the like, or the flow may be governed by one or more extrinsic inputs, e.g., by a controller operative on input signals from sensors that detect pressure or flow at relevant locations (e.g., a pressure transducer in the fluid connection (17)), or biosensors that provide other indicia relevant to selecting the rate for achieving and maintaining the desired drug delivery conditions. In certain embodiments, the apparatus comprises more than one infusion catheter, e.g., to treat independent target tissues.

The invention also encompass the use of one or more sensors that provide output signals upon which the controller operates to determine a pumping, drug release or TCE regimen. The sensors may sense fluid pressure, detect the level or presence of a substance, a drug or a metabolite, electric field strength, tissue temperature or detect a physiologic condition to which the treatment is applied. Advantageously, an array of sensors may themselves be implanted at positions to determine the spatial distribution in the target tissue of the drug delivered by the delivery system, and a processor or controller may operate accordingly to achieve the delivery of the desired dose or concentration distribution, or to achieve the desired control of sensed conditions during changing metabolic and tissue states. The invention also encompasses an apparatus that has presettable procedure parameters, procedure automation, and/or closed loop systemic control. In certain embodiments, the control system and electrical signal generator are incorporated into a portable or handheld unit.

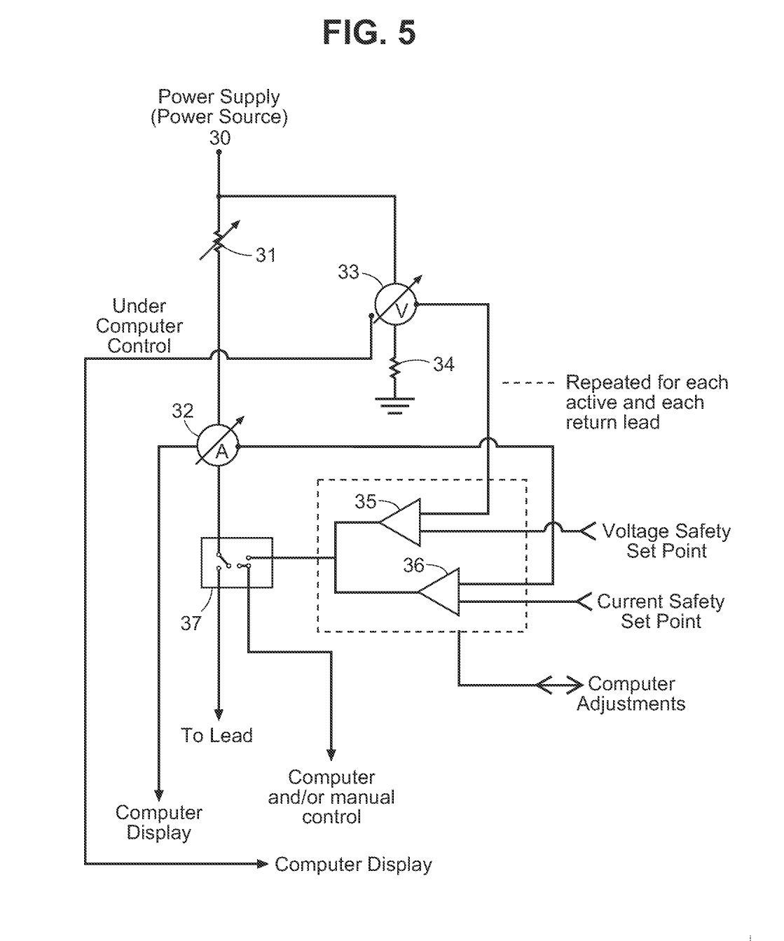

FIG. 5 schematically illustrates an exemplary component of one embodiment of the invention, which embodiment comprises an independent safety circuit for each electrode. The safety circuit comprises a lead to the electrode (37) and a tuning resistor (31) that can be manually or, under control of a processor, set to continually regulate the current flow to the circuit and lead. The circuit also comprises current and voltage sensors ((32) and (33), respectively) that monitor the current flow and voltage in the specific lead/electrode. The output of the current and voltage sensors can be used to set the tuning resistor to modify the current passing from the power supply to the lead/electrode. The circuit may additionally comprise voltage and current safety set points ((35) and (36), respectively) such that a voltage/current outside of a defined range will cut power and/or current to the specific lead and, thus, electrode. The output from any sensor in the circuit may be displayed for operator use or may serve as an input for processor control.

In certain aspects, one or more implantable components of the invention (e.g., one or more electrodes, one or more infusion catheters, one or more integrated TCE cannulas) are provided in a housing or casing to facilitate its handling and/or implantation. The housing or casing is generally made of a biocompatible, sterilizable material and further can comprise one or more activating buttons connected to switches coupled to an interfacing connector for connection to a source device of the apparatus. In certain embodiments, the buttons may be used to manually activate one or more functions of the implanted device (e.g., imaging, drug delivery, electrode operation and the like). In specific embodiments, the housing for the integrated TCE cannula is removed once the cannula is implanted.

The implantable component, or the housing and/or casing thereof, can comprise one or more radiopaque markers. This may be useful, for example, for positioning the component during initial implantation and/or during routine assessments of the apparatus in the case of chronic implantation.

5.4 Therapeutic Applications

The methods of the invention encompass the use of two or more electrodes surrounding or within target tissue such as tissues of the CNS or any other tissue or organ for which administration of an agent, e.g., a therapeutic or diagnostic agent, to a specific region of the tissue or organ is desired and within which an electric field may be generated in accordance with the methods described herein. The placement of the electrodes according to the methods of the invention creates an electric field within or through the target tissue that provides an EMF of sufficient force to effect the movement of an agent to or through the target tissue.

In preferred embodiments, the target tissue comprises brain tissue, and the movement of the agent through the brain tissue in response to the electric field is termed trans-cerebral electrophoresis (“TCE”). In optional embodiments, TCE may be combined with CEDD using one or more infusion catheters or cannulas. In a specific embodiment, when TCE is combined with CEDD, one or more integrated TCE cannulas may be used that separately function as both an infusion catheter and an electrode. As described herein, the three dimensional spacing of two or more electrodes in the electrode array, and the parameters of the electric field developed between them, relative to the site of therapeutic agent application, will determine the volume of agent distribution. The phrase “volume of agent distribution” refers to a region of a solid tissue into which delivery of a therapeutic agent is desired and/or achieved. For example, the volume of distribution may correspond with the volume occupied by a tumor, or may be a particular region of the brain that is targeted for treatment. In certain aspects, the volume of distribution is determined by the use of a tracer compound (e.g., an MRI or X-ray contrast agent) and/or the use of a modeling system (e.g., a mathematical model or an animal model, e.g., cat). The volume of distribution also may be smaller or greater than the tracer's observed volume of distribution, in which case, a correlation between the volume of distribution of the tracer and the volume of distribution of the therapeutic agent may be used to convert the observed tracer distribution to a therapeutic agent distribution. When monitoring the distribution of a tracer, infusion may be stopped when the desired volume of distribution is reached, regardless of the relative mobilities of the tracer and therapeutic agent in the tissue. A determination of whether or not the specific tracer has a mobility that is equivalent to that of an agent to be administered, or a determination of how the volume of distribution of a tracer correlates to the volume of distribution of the desired agent may, for example, be determined by animal studies which compare the volume of distribution of, e.g., a radiolabeled agent (determined, for example, by QAR or PET scanning) to the volume of distribution determined by MRI or CT for a co-infused tracer. In specific embodiments, the tracing agent comprises a liposome. In other embodiments, the tracing agent comprises a liposome containing an MRI contrast agent, e.g., a gadolinium chelate.

TCE is applicable to the delivery of a variety of classes of therapeutic agents for a variety of purposes and may be sustained in cycles lasting several minutes, hours, days, weeks, months, years, or may in some instances be continuous (i.e., chronic treatment). In other embodiments, the apparatus may only be used for relatively short periods, for example, in response to detection of a condition or as a diagnostic tool. In certain embodiments, the apparatus of the invention comprises chronically implanted components but the apparatus is only activated periodically. It will be understood that the precise parameters and duration of activation will depend upon a variety of factors, including the identity and concentration of the agent, drug or bioactive material and carrier, the size and tissue properties of the target site, the nature of the disease or disorder to be treated, and the manner of agent application, e.g., the dosing and total number of cycles of agent administration, e.g., via an infusion catheter.

It is envisioned that the methods of the invention using TCE provide a more homogeneous and far-reaching distribution and/or more directed and controllable administration of an agent than can currently be achieved with CEDD alone. The invention encompasses the delivery of agents that naturally posses or may be chemically modified (without losing their desired bioactive property(ies)) to have sufficient charge to respond to the EMF developed within the tissue according to the methods of the invention. Any such agent, e.g., therapeutic, investigational or diagnostic agent, possessing or capable of being modified to posses (without losing their desired bioactive property(ies)) such EMF responsive characteristics may therefore be used with the apparatus and according to the methods of the invention for the treatment of any CNS disease or disorder and/or for delivery of pharmaceuticals, gene therapies, peptides, proteins or any charged compound for the purpose of conducting diagnostic assays or neuroscience research. Non-limiting examples of such agents include proteins, peptides, polypeptides, neurotrophic factors, gene therapies (both virally and liposomally mediated) and small molecules, and non-limiting examples of such CNS diseases or disorders include malignant or benign tumors of the CNS, amyloidogenic diseases (e.g., Alzheimer's disease), neurodegenerative disorders (e.g., Parkinson's disease), inflammatory disorders (e.g., Multiple sclerosis, Neurosarcoidosis), infections (e.g. encephalitis, HIV cerebritis, PML, tuberculosis), lysosomal storage diseases, mitochondrial diseases or other genetically mediated central nervous system disorders. Additionally, the methods of the invention encompass the treatment of acute CNS diseases or disorders, for example, but not limited to, CNS trauma and stroke.

Treatment according to the methods of the invention may improve the subject's condition to a clinical endpoint, which endpoint may be amelioration of the disease or disorder, complete or partial recovery from the disease or disorder, or reduction or amelioration of one or more symptoms of the disease or disorder. Once the clinical endpoint is reached, treatment according to the methods of the invention may be stopped. However, the methods of the invention also encompass the treatment of chronic diseases or disorders requiring chronic treatment. The methods of the invention for treating a subject can be supplemented with other forms of therapy. Supplementary therapies include drug treatment, radiation therapy, a change in diet, etc. Supplementary therapies can be administered prior to, contemporaneously with or following the invention methods of treatment. The skilled artisan can readily ascertain therapies that may be used in a regimen in combination with the treatment methods of the invention.

Agents that may be used in accordance with the methods of the invention include, but are not limited to, antineoplastic agents, radioiodinated compounds, toxins (including protein toxins), cytostatic or cytolytic drugs, genetic and viral vectors, neurotrophic factors, cytokines, enzymes and agents for targeted lesioning of specific sites. Therapeutic agents also include any therapeutic molecule which is targeted selectively to a cell expressing a particular antigen, for example, antibodies and immunotoxins (see, for example, Laske et al., “Tumor regression with regional distribution of the targeted toxin TF-CRM107 in patients with malignant brain tumors,” Nature Medicine, 3: 1362-1368, 1997). Non-limiting examples of agents that may be used according to the methods of the invention include Doxorubicin, Temozolomide, Carbusin, Carmustine, Bevacizumab, Cisplatin, Nitrosoureas (BCNU, CCNU), anti-angiogenesis factors, therapies targeted to cell-surface receptors, virally and liposomally mediated gene therapies, cDNA, plasmid DNA, RNA including siRNA, toxins directed to tumor antigens via specific antibodies, growth factors such as GDNF, BDNF, NGF, VGF, immunomodulating agents such as interleukins, interferons, antiviral agents, and stem cells. But, as embodied herein, any biologically active compound that is sufficiently charged to respond to the electrical field established by the immediate invention, or may be modified to respond to an electrical field without mitigating its desired biological activity, and is therapeutically or diagnostically useful for the CNS disease, injury, or disorder to be treated or investigated, may be employed.

The specific dose of the one or more therapeutic agent is typically calculated according to the volume of distribution for the particular subject. The calculations necessary to determine the appropriate dosage for treatment involving pharmaceutical formulations is routinely made by those of ordinary skill in the art and is within the ambit of tasks routinely performed by them without undue experimentation.

The course of treatment according to the methods of the invention, e.g., using the TCE apparatus as described herein, may be continuous or may be provided in one or more repeated intervals until the desired therapeutic result or total dose to the target tissue is achieved. Treatment parameters are dictated by the nature of the disease or disorder to be treated, the bioactivity and biodistribution of the agent(s) to be administered and the response of the subject to the treatment. The efficacy of the therapeutic methods of the invention will de determined at intervals determined by the treating clinician. The determination of the appropriate length of treatment or the appropriate number of treatments, and the methods and times of assessment of therapeutic efficacy are routinely made by those of ordinary skill in the art and are within the ambit of tasks routinely performed by them without undue experimentation.

In specific embodiments, a course of treatment with the TCE apparatus according to the methods of the invention is repeated at intervals of about 1-2 days, about 1-4 days, about 1-5 days, about 1 week to about 2 weeks, about 1 week to about 3 weeks, about 1 week to about 4 weeks, about 1 week to about 1 month, about 1 week to about 2 months, about 1 week to about 4 months, about 1 week to about 6 months, about 1 week to about 8 months, about 1 week to about 9 months, about 1 week to about 10 months, about 1 week to about 12 months, about 1 week to about 15 months, about 1 week to about 18 months, about 1 week to about 24 months, about 1 week to about 30 months, or about 1 week to about 36 months. In other embodiments treatment with the TCE apparatus according to the methods of the invention is repeated daily, or at 2 day, 4 day, 5 day, 1 week, 2 week, 3 week, 4 week, 1 month, 2 month, 4 month, 6 month, 8 month, 9 month, 10 month, 12 month, 15 month, 18 month, 24 month, 30 month, or 36 month intervals. The repeat regimen may be administered as a matter of course, for example, when symptoms associated with the CNS disease or disorder recur after an improvement following the initial or previous therapy, or when symptoms associated with the CNS disease or disorder do not improve after the initial therapy according to methods of the invention.

Efficacy of the treatment may be determined as described herein or as is known in the art in between treatment intervals, during continuous treatment or after cessation of treatment according to the methods of the invention. Determinations of treatment efficacy will be made by the treating clinician according to standard practices in the art. In certain embodiments, the diagnostic determinations of treatment efficacy may be made at 1 hour, 2 hour, 5 hour, 12 hour, 24 hour, 48 hour, 72 hour, 96 hour, 110 hour, 1 week, 2 weeks, 3 weeks, 4 weeks, 1.5 months, 2 months, 4 months, 6 months, 9 months, 12 months, 15 months, 18 months, 24 months, 30 months, or 36 months after the start of treatment according to the methods of the invention. In other embodiments the diagnostic determination of treatment efficacy may be made in between treatments, for example at 1 week, 2 weeks, 3 weeks, 4 weeks, 1.5 months, 2 months, 4 months, 6 months, 9 months, 12 months, 15 months, 18 months, 24 months, 30 months, or 36 months subsequent to the initial or previous treatment or 1 week, 2 weeks, 3 weeks, 4 weeks, 1.5 months, 2 months, 4 months, 6 months, 9 months, 12 months, 15 months, 18 months, 24 months, 30 months, or 36 months prior to the beginning of the next course of treatment.

In another embodiment, the subject is provided TCE therapy according to the methods of the invention wherein the therapy is continuous administration over about 1-2 hours, 1-4 hours, 1-6 hours, 1-8 hours, 1-12, hours, 1-18 hours, 1 hour to 1 day, 1 hour to 2 days, 1 hour to 3 days, 1 hour to 4 days, 1 hour to 5 days, 1 hour to 6 days, 1 hour to 7 days, 1 hour to 8 days, 1 hour to 9 days, 1 hour to 10 days, 1 hour to 11 days, 1 hour to 12 days, 1 hour to 13 days or 1 hour to 14 days. In other embodiments, the treatment with the TCE apparatus and according to the methods of the invention is continuous administration over about 30 min, 1 hour, 2 hours, 4 hours, 6 hours, 8 hours, 10 hours, 12 hours, 18 hours, 1 day, 2 days, 3 days, 4 days, 5 days, 6 days, 7 days, 8 days, 9 days, 10 days, 11 days, 12 days, 13 days or 14 days. In certain embodiments, the TCE therapy may be chronic.

In other embodiments, the invention provides for dose escalation, wherein the TCE therapy comprises increasing the dose of the therapeutic agent until the daily prophylactically or therapeutically effective amount of the therapeutic agent is achieved. Depending on the therapeutic agent, it may be desirable to increase the effective amount of the agent over time until the therapeutically or prophylactically total dose is achieved. For example, in certain embodiments, the dose of administered agent escalates over the first fourth, first half or first ⅔ of the treatment regimen. In other embodiments, a subject is administered a treatment regimen comprising an infusion of a therapeutic agent, wherein the prophylactically or therapeutically effective amount of the agent is increased by a factor of 1.25, a factor of 1.5, a factor of 2, a factor of 2.25, a factor of 2.5, or a factor of 5 per hour or day until the daily prophylactically or therapeutically effective amount, or until the total desired dose, of the agent is achieved.

5.5 Pharmaceutical Compositions

The present invention provides compositions comprising therapeutic agents for the treatment, prophylaxis, and amelioration of one or more symptoms associated with a disease or disorder of the CNS. In certain embodiments, in addition to one or more therapeutic agents, the composition may also comprise an agent for modifying osmotic pressure in vivo and/or facilitating movement of the therapeutic agent, e.g., mannitol.

As recognized in the art, in certain embodiments, buffers, emulsifying agents or diluents may be required for the preparation of the therapeutic infusion. For example, emulsifying agents may be required in order to modify uncharged lipophilic compounds such that the compounds and/or resultant composition develop(s) a sufficient charge to be deliverable according to the methods of the invention. However, tissue reactions with the buffers, diluents and/or emulsifying agents should be considered and those with potentially toxic properties avoided. In preferred embodiments, the diluent is saline and/or glucose. In certain embodiments, the composition for use according to the methods of the invention is phosphate buffered.

In certain embodiments, the composition for use in accordance with the methods of the invention is a pharmaceutical composition. Such compositions may comprise a prophylactically or therapeutically effective amount of one or more therapeutic agents for the treatment of CNS diseases or disorders, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. In preferred embodiments, the carrier is suitable for administration to CNS tissue, e.g., the brain. Saline solutions are the preferred carriers, optionally comprising dextrose and glycerol, e.g., for modifying the viscosity of the composition.

The composition, if desired, can also contain minor amounts of wetting or emulsifying agents, or pH buffering agents, provided that the agents are suitable for use with the target tissue. These compositions can take the form of solutions, suspensions, emulsions and the like. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient, e.g., via CEDD. In a preferred embodiment, the pharmaceutical compositions are sterile and in suitable form for CEDD administration to a subject, preferably an animal subject, more preferably a mammalian subject, and most preferably a human subject.

In specific embodiments, the pharmaceutical composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### 6. EXAMPLES

### 6.1 Glioblastoma

A patient is diagnosed with a malignant tumor involving the basal ganlia of the right cerebral hemisphere. Biopsy indicates that the tumor is a glioblastoma multiforme. The risks of open resection of the tumor are great and the tumor is too large for stereotactic radiosurgery.

In a surgical setting, a combination infusion catheter/central electrode is stereotactically inserted into the center of the tumor and a series of burr holes are made at various sites along the ipsilateral hemicalvarium through which are inserted plate electrodes. The electrodes are left resting on the surface of the brain and are similar to the recording electrodes commonly employed for invasive EEG monitoring. The infusion catheter and the wires connected to the electrode array exit the scalp via sites other than the incision created to insert the various components. Incisions are closed according to standard surgical techniques.

The infusion catheter is primed with a solution containing the therapeutic agent and is connected to a convective infusion pump, which is programmed to deliver the agent at a specified rate/dose. The electrode wires are connected to an electrical generator, which will create the electrical field down which the therapeutic agent will migrate. Polarity of the central electrode, i.e., that of the combination infusion catheter/electrode, and the electrode array is set to effect dispersion of the therapeutic agent according to the charge profile of the agent. For negative agents, the central electrode is set as the negative pole (i.e., the cathode) and the surface electrodes are set as positive poles (i.e., the anodes). The parameters of the electrode array are programmed into the electrical generator and therapy is initiated.

The combination convective infusion and electrical gradient distribute the therapeutic agent throughout the tumor and the surrounding white matter over the course of hours to a few days. The agent may be a chemotherapy encapsulated in nanospheres that are designed to release the chemotherapeutic agent slowly into the brain parenchyma over the ensuing weeks. The therapeutic agent is mixed with a tracer molecule such at gadolinium to enable evaluation of the distribution of the delivered compound. On completion of the therapy, the electrode wires and infusion catheter are disconnected from the TCE machine, electrical generator and any other accessory devices according to the methods of the invention and the patient is returned to the surgical setting for removal of the electrodes and catheter.

### 6.2 Alzheimer's Disease

A patient diagnosed with Alzheimer's disease is admitted to the hospital for TCE therapy. In a surgical setting one or more combination infusion catheters/central electrodes are inserted into the patient's frontal lobes bilaterally. A series of burr holes are made at various sites around the calvariusm through which are inserted plate electrodes. The electrodes are left resting on the surface of the brain and are similar to the recording electrodes commonly employed for invasive EEG monitoring. The catheters are primed with a solution containing a therapeutic neurotrophic compound and connected to tubing tunneled under the skin to the abdomen where the tubing is connected to a programmable convective pump. The pump is or has been previously implanted within a subcutaneous pocket created by a surgeon. The electrode wires are brought to a central point at the scalp and connected to an extension cable. Similar to the catheter tubing, the extension cable is tunneled subcutaneously to a multichannel, programmable stimulator that is placed within a subcutaneous pocket at the chest wall. All incisions are closed according to standard surgical techniques.

After discharge and a time sufficient for healing of the surgical/incision sites, the devices are activated. The pump is programmed to deliver the therapeutic compound at the desired rate and the generator is programmed to created the desired electrical gradient. The therapeutic reservoir and remaining power may be monitored by any trained medical staff and refilled in a hospital setting or, alternatively, in the patient's home by a trained health care provider.

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

What is claimed:

1. An implantable, integrated TCE cannula for delivery of an agent to or within tissue of a subject in need thereof, said cannula comprising
(a) a cannula having proximal and distal ends, wherein said cannula provides a fluid delivery pathway for fluid when said proximal end of the cannula is connected to a fluid delivery system,
(b) one or more outlet ports at the distal of said cannula through which the agent exits said cannula from the fluid delivery pathway for administration to the tissue, and
(c) a monopolar electrode having a region for connecting to an electrical power source.

2. The integrated TCE cannula of claim 1, wherein the proximal end of the cannula comprises a connector for connecting said fluid delivery pathway and said fluid delivery system, and wherein said connector is suitable for temporary or permanent connection.

3. The integrated TCE cannula of claim 1, wherein the region for connecting the monopolar electrode and electrical power source comprises a connector for connecting said electrode and said power source, and wherein said connector is suitable for temporary or permanent connection.

4. The integrated TCE cannula of claim 1, further comprising a thermocouple in connection with an output display device or a processor that regulates power supply to the monopolar electrode.

5. The integrated TCE cannula of claim 1, wherein the fluid delivery pathway is primed with a pharmaceutically acceptable carrier or with a pharmaceutical composition.

6. An apparatus for delivery of an agent to a target tissue of a subject, said apparatus comprising
(a) an implantable, integrated TCE cannula comprising
(i) a cannula having proximal and distal ends, wherein said cannula provides a fluid delivery pathway for fluid when said proximal end of the cannula is connected to a fluid delivery system,
(ii) one or more outlet ports at the distal of said cannula through which the agent exits said cannula from the fluid delivery pathway for administration to the tissue, and
(iii) a monopolar electrode having a region for connecting to an electrical power source.
(b) at least one monopolar electrode separate from that of the implantable, integrated TCE cannula having a region for connecting to an electrical power source;
wherein connection of the at least two electrodes to the power source polarizes the electrodes, creating an electric field between them.

7. The apparatus of claim 6, wherein said integrated TCE cannula comprises, at its proximal end, a connector for connecting said fluid delivery pathway and said fluid delivery system, and wherein said connector is suitable for temporary or permanent connection.

8. The apparatus of claim 6, wherein the integrated TCE cannula comprises, at its proximal end a connector for connecting said electrode and said power source, which connector is located in said region for connecting the monopolar electrode and electrical power source, and wherein said connector is suitable for temporary or permanent connection.

9. The apparatus of claim 6, wherein the apparatus comprises a plurality of monopolar electrodes separate from that of the implantable cannula.

10. The apparatus of claim 9, wherein the plurality of the monopolar electrodes is a plurality of surface electrodes, a plurality of implantable electrodes or a plurality of surface and implantable electrodes, and wherein their spatial arrangement is such that the developed electric field between the electrodes at least partially encompasses the target tissue.

11. The apparatus of claim 6 further comprising a fluid delivery system.

12. The apparatus of claim 6 further comprising a power source.

13. The apparatus of claim 9 further comprising a power source.

14. The apparatus of claim 12 or 13 further comprising a processor in electrical communication with the two or more monopolar electrodes and the power source, which processor regulates the power supply to the two or more monopolar electrodes.

15. The apparatus of claim 11 or 13 further comprising a processor in electrical communication with the fluid delivery system, which processor regulates the rate of agent or fluid flow from the fluid delivery system.

16. The apparatus of claim 13 further comprising a processor in electrical communication with the fluid delivery system, the two or more monopolar electrodes and the power source, which processor regulates the power supply to the two or more electrodes and regulates the rate of agent or fluid flow from the fluid delivery system.

17. The apparatus of claim 6 further comprising a thermocouple in connection with an output display device or a processor that regulates power supply to the two or more monopolar electrodes.

18. The apparatus of claim 17, wherein said thermocouple is a component of said implantable cannula.

19. The apparatus of claim 12, wherein said source provides a direct current.

20. The apparatus of claim 12, wherein said power source provides an alternating current.

21. The apparatus of claim 19 or 20, wherein the current is pulsed.

22. The apparatus of claim 21, wherein the pulsed current is delivered for a duration of about 1 microsecond to 5 seconds provided at a frequency of from 0 to 10 Hertz.

23. The apparatus of claim 11, wherein said fluid delivery system comprises a pump capable of delivering fluid at a constant or variable flow rate.

24. The apparatus of claim 11 or 23, wherein said fluid delivery system comprises multiple separate fluid reservoirs each in liquid communication with the implantable cannula.

25. The apparatus of claim 23, wherein the fluid delivery system is capable of delivering fluid at a rate of 0.1 μl/hr to 25 μl/min.

26. The apparatus of claim 6, wherein the apparatus further comprises one or more biosensors in communication with a processor that regulates the operating parameters of the apparatus in response to signals from the biosensor.

27. A method for delivering an agent to a target tissue of a subject in need thereof, said method comprising:
(a) positing an array of at least two electrodes within or external to the subject,
(b) implanting a cannula in the subject to deliver said agent to the agent delivery site,
(c) infusing said agent through the cannula to the agent delivery site, and
(d) polarizing the array of electrodes thereby generating the electric filed within the array,
wherein said agent is responsive to an EMF, wherein the spatial arrangement of the electrodes causes the target tissue to be at least partially encompassed by the electric field, and wherein the electric field disperses the agent along the developed electric potential gradient and toward, within, or throughout the target tissue.

28. The method according to claim 27, wherein said tissue is CNS tissue or within the CNS tissue of the subject.

29. The method of claim 27, wherein the agent is a therapeutic agent, diagnostic agent, an investigational agent or a pharmaceutical composition.

30. A method of treating a CNS disease or disorder, said method comprising administering a therapeutically effective amount of an agent therapeutic for the disease or disorder to a target tissue in a subject in need thereof by
(a) positing an array of at least two electrodes within or external to the subject,
(b) implanting a cannula in the subject to deliver said agent to the agent delivery site,
(c) infusing said agent through the cannula to the agent delivery site, and
(d) polarizing the array of electrodes thereby generating the electric filed within the array,
wherein said agent is responsive to an EMF, wherein the spatial arrangement of the electrodes causes the target tissue to be at least partially encompassed by the electric field, and wherein the electric field disperses the agent along the developed electric potential gradient and toward or within the target tissue.

31. The method according to claim 28 or 30, wherein the target tissue is the brain or spinal cord.

32. The method according to claim 27 or 30, wherein said cannula is an integrated TCE cannula comprising
(a) a cannula having proximal and distal ends, wherein said cannula provides a fluid delivery pathway for fluid when said proximal end of the cannula is connected to a fluid delivery system,
(b) one or more outlet ports at the distal of said cannula through which the agent exits said cannula from the fluid delivery pathway for administration to the tissue, and
(c) a monopolar electrode having a region for connecting to an electrical power source.

33. The method according to claim 32, wherein the array of electrodes is implanted in the subject and the monopolar electrode of the integrated TCE cannula is one of the at least two electrodes of said electrode array.

34. The method of claim 30, wherein the CNS disease or disorder is a CNS cancer or malignancy, a neurodegenerative disorder, an amyloidogenic disease, a condition or symptom associated with stroke, a mitochondrial disorder, an inherited disorder, a traumatic or hypoxic brain injury, a birth-related injury, an infection, HIV, or a lysosomal storage disease.

35. The method according to claim 27 or 30 wherein the agent delivery site is not within the target tissue or within the developed electric field.

36. A method for delivering an agent to a target tissue within the CNS of a subject, said method comprising the steps of
(a) positioning an array of electrodes within the CNS tissue of the subject;
(b) polarizing the array of electrodes thereby generating an electric field between the electrodes; and
(c) applying the agent within the electric field,
wherein the spatial arrangement of the electrodes causes the target tissue to be at least partially encompassed by the electric field, and wherein the electric field disperses the therapeutic agent along the developed electric potential gradient and toward the target tissue.

37. A method of treating a CNS disease or disorder, said method comprising
(a) positioning an array of electrodes within the CNS tissue of a subject in need thereof;
(b) polarizing the array of electrodes thereby generating an electric field between the electrodes; and
(c) administering a therapeutically effective amount of an agent therapeutic for said disease or disorder to said subject and within the electric field,
wherein the spatial arrangement of the electrodes causes the target tissue to be at least partially encompassed by the electric field, and wherein the electric field disperses the therapeutic agent along the developed gradient and distributing it to and/or within the target tissue.

38. The integrated TCE cannula of claim 1, wherein said tissue is CNS tissue or within the CNS tissue of the subject.

39. The apparatus of claim 6, wherein the fluid delivery pathway of the implantable, integrated TCE cannula is primed with a pharmaceutically acceptable carrier or with a pharmaceutical composition.
